(19) United States
(12) Patent Application Publication
(10) Pub. No.: US 2016/0022675 A1
(43) Pub. Date: Jan. 28, 2016

(54) PROCESSES FOR PREPARING TETRAHYDROISOQUINOLINES

(71) Applicant: BRISTOL-MYERS SQUIBB COMPANY, Princeton, NJ (US)

(72) Inventors: Francisco GONZALEZ-BOBES, New Brunswick, NJ (US); David Alan CONLON, New Brunswick, NJ (US); Paul C. LOBBEN, New Brunswick, NJ (US); Justin Lockheart BURT, Greenwood, IN (US); Joshua ENGSTROM, New Brunswick, NJ (US); Carolyn S. WEI, New Brunswick, NJ (US); Jason J. ZHU, New Brunswick, NJ (US); Yu FAN, New Brunswick, NJ (US)

(21) Appl. No.: 14/774,522

(22) PCT Filed: Mar. 12, 2014
(86) PCT No.: PCT/US2014/023938

§ 371 Date: Sep. 10, 2015

Related U.S. Application Data
(60) Provisional application No. 61/782,359, filed on Mar. 14, 2013.

(51) Int. Cl. A61K 31/501; A61K 9/20; A61K 9/48; C07D 217/04

(52) U.S. Cl. CPC A61K 31/501; C07D 217/04; A61K 9/20; A61K 9/48

(57) ABSTRACT

Disclosed are processes for preparing tetrahydroisoquinolines, intermediates useful in the preparation of tetrahydroisoquinolines, processes for preparing such intermediates, and compositions comprising the tetrahydroisoquinolines and other compounds, e.g, intermediates and by-products of the processes described herein. Pharmaceutical compositions comprising tetrahydroisoquinolines, methods of using tetrahydroisoquinolines in the treatment of depression are also disclosed.

### CROSS REFERENCE TO RELATED APPLICATION

This application claims the priority of U.S. Provisional Application Ser. No. 61/782,359 filed Mar. 14, 2013 which is herein incorporated by reference in its entirety.

The present invention generally relates to processes for preparing tetrahydroisoquinolines, intermediates useful in the preparation of tetrahydroisoquinolines, processes for preparing such intermediates, and compositions comprising the tetrahydroisoquinolines and other compounds, e.g, intermediates and by-products of the processes described herein. The present invention also generally relates to pharmaceutical compositions comprising tetrahydroisoquinolines, methods of using tetrahydroisoquinolines in the treatment of diseases such as, for example, depression.

Major depressive disorder (MDD) exerts a devastating impact on the individual, their families and society in general. The World Health Organization (WHO) estimates that MDD will be the second leading cause of disability by the year 2020. MDD is characterized by diverse symptoms that include depressed mood, suicidality, psychomotor retardation or agitation, reduced motivation or hopelessness, and anhedonia (Kessler R C, Chiu W T, Demler O, Walters E E. Prevalence, severity, and comorbidity of 12-month DSM-IV disorders in the national comorbidity survey replication. Arch. Gen. Psychiatry. 2005; 62:617-627 (2005); erratum). MDD is a debilitating disease, eroding quality of life, productivity in the workplace, and fulfillment of social and familial roles. MDD causes a large amount of non-fatal disease burden, accounting for almost 12% of all total years lived with disability worldwide (Ustun T B, Aryuso-Mateos J L, Chatterji S, et al. Global burden of depressive disorders in the year 2000. Br J Psychiatry. 2004; 184:386-392). With a 12-month prevalence rate of more than 5%, the treatment costs of MDD are soaring but are only a fragment of the costs of decreased productivity due to depression (Smit F, Willemse G, Koopmanschap M, Onrust S, Cuijpers P, Beekman A. Cost effectiveness of preventing depression in primary care patients: randomised trial. Br J Psychiatry. 2006; 188:330-336). With all its concomitant economic costs to society, MDD ranks third among disorders responsible for global disease burden, and may rank first in high-income countries by 2030 (Mathers C D, Loncar D. Projections of global mortality and burden of disease from 2002 to 2030. PLoS Med. 2006; 3(11):e442). After a first episode of MDD, many patients develop a recurrent or chronic disorder and are likely to spend a substantial proportion of their lifetime in a depressed condition. MDD can lead to considerable additional damage through biological sequelae and maladaptive illness behaviors, with increased risk of cardiovascular disease, dementing illnesses and early death, and amplification of disability, complications, and health care utilization in those with coexisting chronic illnesses.

Despite the variety of treatment options, restoring patients with MDD to health remains challenging. As described in the largest naturalistic study of depression, the National Institute of Mental Health funded Sequenced Treatment Alternatives to Relieve Depression (STAR*D) trial, a significant proportion of patients are unresponsive to multiple treatment trials (Warden D, Rush A J, Trivedi M H, Fava M, Wisniewski S R. The STAR*D Project results: A comprehensive review of findings. Current Psychiatry Reports. 2007; 9:449-459). Only about one-third of the participants reached remission after receiving the first treatment trial, which was citalopram as monotherapy (Rush A J, Warden D, Wisniewski S R, Fava M, Trivedi M H, Gaynes B N, Nierenberg A A. STAR*D: revising conventional wisdom. CNS Drugs. 2009; 23(8):627-647). Those who did not remit were advanced to additional treatment trials. Remission rates were similar for the second trial, but were approximately 15% for the third and fourth trials. Over the course of all four treatment trials, almost 70% of patients who did not withdraw from the study entered remission (Rush et al, 2009). However, if the rate at which participants withdrew from the trial is considered, 20% after the first trial, 30% after the second trial, and 43% after the third trial, only 45% of initial enrollees achieved remission. Even with response to treatment, the incidence of relapse is high and the likelihood of relapse is further exacerbated by an inadequate duration of maintenance treatment, often as a result of adverse effects such as sexual dysfunction, weight gain, and sleep disturbance.

Thus, there is substantial unmet medical need for new treatments that can improve outcomes for patients with inadequate response to previous antidepressants in the current episode. Desirably, improved outcomes would encompass rapid remission and sustained symptom improvement over the longer term.

Symptoms of a major depressive episode that remain after treatment with commonly used serotonergic or serotonergic and norepinephrinergic antidepressants—whether due to failure to respond, to remit or to achieve asymptomatic status may often be related to inability to maintain drive and motivation. These abilities require a functioning dopaminergic system and may underlie a broad range of symptoms, including feelings of happiness (joy), interest, energy, enthusiasm, alertness, self-confidence and psychomotor slowing. This “negative symptom” dimension is most prominently impaired in melancholic depression but may also be involved in non-melancholic depression. Therefore, it is important to identify symptoms and symptom clusters that predict poor outcome to widely used antidepressants such as selective serotonin reuptake inhibitors (SSRIs), dual serotonin and norepinephrine reuptake inhibitors (SNRIs), and tricyclic antidepressants (TCAs). Due to the purported role of dopamine in the pathophysiology of depression, triple reuptake inhibitors (TRIs) that simultaneously inhibit serotonin (5-HT), norepinephrine (NE) and dopamine (DA) reuptake could be the next generation of drugs for the treatment of major depression. While TRIs may be useful in managing non-melancholic depression that has not responded adequately to these agents, they may be first line treatment for melancholic depression. The balance of monoamine transporter inhibitory activity across the dose range may be key to the effectiveness of these agents.

Accordingly, there is a need for increased efficacy over SSRIs and SNRIs. In addition, reduction in the degree of side effects is also desired. For example, addressing decreased libido as well as sleep and gastrointestinal disorders (GI) disorders associated with current anti-depressants would provide substantial benefits to patients.

There is also a need to provide compositions comprising tetrahydroisoquinolines that have defined amounts of intermediates and by-products resulting from the processes used to make the tetrahydroisoquinolines. Excessive amounts of such intermediates and by-products may adversely impact the product quality of the tetrahydroisoquinolines, or their stability or their safety characteristics, i.e., increased toxicity levels.

It is desired to provide compositions containing tetrahydroisoquinolines as drugs that target the DAT, serotonin transporter (SERT), and norepinephrine transporter (NET). Desirably, the drugs would provide a desired ratio of SERT, DAT and NET inhibition. Accordingly, SERT, DAT and NET occupancies are important pharmacological criteria for consideration. In one aspect of the invention, compounds are provided that lead to greater than about 10% occupancy, e.g., 10-40%, while maintaining SERT occupancy greater than about 60%, e.g., 60-80%.

It is also desired to provide processes for making tetrahydroisoquinolines, intermediates useful in the preparation of tetrahydroisoquinolines, compositions comprising the tetrahydroisoquinolines and other compounds, e.g, intermediates and by-products of the processes described herein, and processes for preparing such intermediates.

It is also desired to provide new methods of treating diseases that are responsive to TRIs, such as, for example, MDD, substance abuse, eating disorders and other conditions. It is especially desired to provide drugs, e.g., compounds of the invention, as an effective monotherapy in the treatment of a major depressive episode (MDE) in MDD patients with a history of an inadequate response to an adequate dose and duration of two different antidepressant classes within the current episode.

It is also desired to provide methods of treatment and pharmaceutical compositions containing tetrahydroisoquinolines, e.g., compounds of the invention such as, for example, Compound 1-(4S) described below, that provide effective oral bioavailability to treat patients.

The inventions described herein are presented in various aspects as set forth below.

The present invention provides processes for making tetrahydroisoquinolines. Also, the present invention provides intermediates useful in the preparation of the tetrahydroisoquinolines. Also, the present invention provides processes for preparing such intermediates. Also, the present invention provides compositions comprising the tetrahydroisoquinolines and other compounds, e.g, intermediates and by-products of the processes described herein.

The present invention also provides methods of treating diseases that are responsive to TRIs, such as, for example, MDD, substance abuse, eating disorders and other conditions. In one aspect, the invention provides drugs, e.g., compounds of the invention, as an effective monotherapy in the treatment of a major depressive episode (MDE) in MDD patients with a history of an inadequate response to an adequate dose and duration of two different antidepressant classes, e.g., duloxetine and escitalopram, within the current episode.

Also, the present invention provides methods of treatment and pharmaceutical compositions containing tetrahydroisoquinolines, e.g., compounds of the invention such as, for example, Compound 1-4(S) described below, that provide effective oral bioavailability, e.g., to provide a total blood plasma concentration profile of the respective compound of the invention, as measured by AUC at 24 hours after an initial dose of the composition, that is at least greater than about 50% of the total blood plasma concentration as measured by AUC at 24 hours of an initial dose of an orally administered solution comprising the respective compound, and/or a blood plasma concentration profile after an initial dose of the composition with a Cmax of Compound 1-(4S) after an initial dose of the composition that is at least greater than about 40% of the Cmax of an orally administered solution comprising Compound 1-(4S).

### DEFINITIONS

Stereochemical definitions and conventions used herein generally follow McGraw-Hill Dictionary of Chemical Terms, S. P. Parker, Ed., McGraw-Hill Book Company, New York (1984) and Stereochemistry of Organic Compounds, Eliel, E. and Wilen, S., John Wiley & Sons, Inc., New York (1994). Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory and (+) or d, meaning the compound, is dextrorotatory. For a given chemical structure, these compounds, called stereoisomers, are identical except that they are mirror images of one another. A specific stereoisomer of a mirror image pair may also be referred to as an enantiomer, and a mixture of such isomers is often called an enantiomeric mixture.

As used herein, the term “alkyl” refers to a straight or branched, saturated aliphatic radical containing one to ten carbon atoms, unless otherwise indicated e.g., alkyl includes methyl, ethyl, propyl, isopropyl, butyl, sec-butyl, isobutyl, tert-butyl, and the like. The term “lower alkyl” refers to an alkyl radical having from one to four carbon atoms.

The term “aryl” refers to a monocyclic or fused bicyclic ring assembly containing 6 to 10 ring carbon atoms wherein each ring is aromatic e.g., phenyl or naphthyl.

The term “heteroaryl” refers to an “aryl” group as defined above, having at least one O, S and/or N interrupting the carbocyclic ring structure, such as pyridyl, furyl, pyrrolyl, thienyl, isothiazolyl, imidazolyl, benzimidazolyl, tetrazolyl, pyrazinyl, pyrimidyl, quinolyl, isoquinolyl, isobenzofuryl, benzothienyl, pyrazolyl, indolyl, isoindolyl, purinyl, carbazolyl, isoxazolyl, thiazolyl, oxazolyl, benzthiazolyl or benzoxazolyl.

The term “substituents” refers to an additional substituent group selected from halogen (preferably fluoro, chloro, or bromo), hydroxy, amino, mercapto, and the like. Preferred substituents for the groups described herein as substituted lower alkyl or substituted alkyl are halogens, particularly fluoro substituents.

The term “hydroxyl protecting group” refers to a protecting group for the —OH moiety when the —OH would otherwise be attached to an alkyl, aryl or amine group. Examples of suitable hydroxyl protecting groups can be found in, for example, Greene and Wuts, PROTECTIVE GROUPS IN ORGANIC SYNTHESIS, 4th edition, John Wiley & Sons, New York, N.Y. (2007).

The term “amine protecting group” refers to a protecting group for the amine moiety. In this instance, the amine group can be attached to an alkyl or aryl moiety or can be present as part of an amide or hydroxamide functional group. Examples of suitable amine protecting groups can be found in, for example, Greene and Wuts, ibid.

The term “removable protecting group” or “protecting group” refers to any group which when bound to a functionality, such as the oxygen atom of a hydroxyl or carboxyl group or the nitrogen atom of an amine group, prevents reactions from occurring at these functional groups and which protecting group can be removed by conventional chemical or enzymatic steps to reestablish the functional group. The particular removable protecting group employed is not critical. Typical protecting groups for use in accordance with the present invention include, but are not limited to, methoxybenzyl, Boc; COOAllyl, COObenzyl, COH, COMe, COPh, and COCF₃.

The term “chiral” refers to molecules which have the property of non-superimposability of the mirror image partner, while the term “achiral” refers to molecules which are superimposable on their mirror image partner.

The term “derivative” means a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

The term “enantiomers” refers to two stereoisomers of a compound which are non-superimposable mirror images of one another.

The term “diastereoisomers” refers to stereoisomers that are not mirror images of one another and are non-superimposable on one another.

The term “regioisomers” refers to molecules with the same molecular formula but that are bonded together in different orders. The term “halogen” as used herein and in the claims is intended to include fluorine, bromine, chlorine and iodine while the term “halide” is intended to include fluoride, bromide, chloride and iodide anion.

The term “patient” includes both human and other mammals.

The term “pharmaceutical composition” means a composition comprising a therapeutic agent in combination with at least one additional pharmaceutically acceptable additive, e.g., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Ingredients listed in Remington's Pharmaceutical Sciences, 18th ed., Mack Publishing Company, Easton, Pa. (1999) for example, may be used.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable risk/benefit ratio.

The term “pharmaceutically acceptable salt” is intended to include nontoxic salts synthesized from a compound which contains a basic or acidic moiety by conventional chemical methods. Generally, such salts can be prepared by reacting the free acid or base forms of these compounds with a stoichiometric amount of the appropriate base or acid, respectively, in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445. Suitable inorganic bases such as alkali and alkaline earth metal bases include The term “polymorph” refers to crystalline forms having the same chemical composition but different spatial arrangements of the molecules, atoms, and/or ions forming the crystal.

The terms “racemic mixture” and “racemate” refer to an equimolar mixture of two enantiomeric species, devoid of optical activity. In addition, as used herein, the terms “racemic mixture” and “racemate” are intended to include equimolar mixtures of two enantiomers.

The term “solvate” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, and the like.

The term “stereoisomers” refers to compounds which have identical chemical constitution, but differ with regard to the arrangement of the atoms or groups in space.

The term “therapeutically effective amount” means the total amount of the therapeutic agent, e.g., tetrahydroisoquinoline, that is sufficient to show a patient benefit. When applied to an individual active ingredient, administered alone, the term refers to that ingredient alone. When applied to a combination, the term refers to combined amounts of the active ingredients that result in the therapeutic effect, whether administered in combination, serially or simultaneously. If, for example, Compound 1-(4S) or form N-1 is used in combination with another medication, i.e., drug, the combination of compounds described herein may result in a synergistic combination. Synergy, as described for example by Chou and Talalay, Adv. Enzyme Regul. 1984, 22, 27-55, occurs when the effect of the compounds when administered in combination is greater than the effect of the compounds when administered alone as single agents.

The term “treating” refers to: (i) preventing a disease, disorder or condition from occurring in a patient which may be predisposed to the disease, disorder and/or condition but has not yet been diagnosed as having it; (ii) inhibiting a disease, disorder or condition, i.e., arresting its development; and (iii) relieving a disease, disorder or condition, i.e., causing regression of the disease, disorder and/or condition.

The phrase “under conditions effective to promote the formation” refers to the reaction conditions, e.g., temperature, pressure, time, state, for example, batch or steady state, liquid or gas, catalysts, reagents, and the like. Typical temperatures range from about −78° C. to 150° C. Typical pressures range from about 0-1500 psig. Typical reaction times range from about 0.5 to 48 hours. Typical states of reaction are liquid phase or gas phase and either batch or steady state in the presence of a catalyst. Specific conditions that are suitable conditions effective to promote the formation of compounds disclosed herein can readily be determined by those skilled in the art.

Unless otherwise noted, the starting materials suitable for use in accordance with the present invention, e.g., raw materials and chemicals, are generally available from commercial sources and otherwise can readily be prepared by those skilled in the art.

For purposes of clarification, the compound 6-[(4)-2-methyl-4-(naphthyl)-1,2,3,4-tetrahydroisoquinolin-7-yl]pyridazin-3-amine, also referred to as 6-[(4)-1,2,3,4-tetrahydro-2-methyl-4-(2-naphthalenyl)-7-isoquinolinyl]-3-pyridazinamine, represented by Formula (I) is referred to herein as Compound 1, and is intended to represent the compound in any of its racemic, enantiomeric or crystalline forms. When Compound 1 is intended to be referred to in its specific racemic form, i.e., free base racemate of rac-6-[(4)-2-methyl-4-(naphthyl)-1,2,3,4-tetrahydroisoquinolin-7-yl]pyridazin-3-amine, the notation Compound 1-rac is use herein. Similarly, the specific (45)-enantiomer of Compound 1, represented by Formula (II), i.e., 6-[(45)-1,2,3,4-tetrahydro-2-methyl-4-(2-naphthalenyl)-7-isoquinolinyl]-3-pyridazinamine or, 6-[(4S)-1,2,3,4-tetrahydro-2-methyl-4-(2-naphthalenyl)-7-isoquinolinyl]-3-pyridazinamine, is referred to herein, in general (without reference to any specific crystalline form), as Compound 1-(4S). Likewise, a specific crystalline form of Compound 1-(4S) in accordance with the present invention, also represented by Formula (II), is referred to herein as Form N-1.

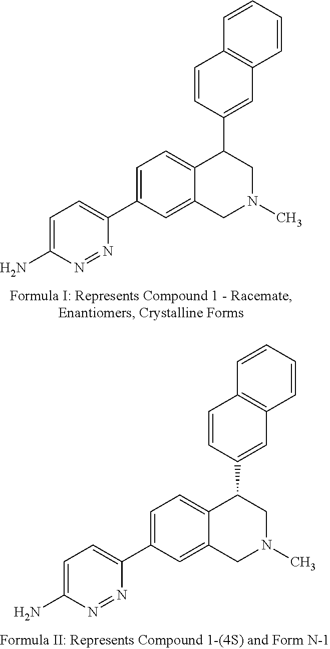 [SMILES: CN1Cc2cc(-c3ccc(N)nn3)ccc2C(c2ccc3ccccc3c2)C1 ; CN1Cc2cc(-c3ccc(N)nn3)ccc2[C@H](c2ccc3ccccc3c2)C1]

Further details concerning the preparation and characterization of Form N-1 are disclosed in WO2009/149258, published Dec. 10, 2009, the contents of which are hereby incorporated by reference.

For ease of reference, the following abbreviations when used in this invention have the meanings given below:

ACN=acetonitrile;
AcOH and HOAc=acetic acid;
Boc=t-butoxycarbonyl;
Boc₂O=di-tert-butyl-dicarbonate
DCM=dichloromethane;
D-DTTA=(2S,3S)-2,3-bis((4-methylbenzoyl)oxy)succinic acid or di-p-toluoyl-D-tartaric acid

### DMAP=4-N,N-dimethylaminopyridine;

### DMF=N,N-dimethylformamide;

DMSO=Dimethyl sulfoxide
e.e. =enantiomeric excess;
EtOH=ethanol;
FeCl3=Iron (III) chloride;
InCl3=Indium (III) chloride;
IPA=isopropanol;
IPAc=isopropyl acetate;
KOAc=potassium acetate;
MeOH=methanol;
MSA=methanesulfonic acid; NaOH=sodium hydroxide
TFA=trifluoroacetic acid;
THF=tetrahydrofuran;
TiCl4=titanium (IV) chloride;
ZrCl4=zirconium (IV) chloride;
g=grams;
h=hours;
kg=kilograms;
L=liters;
min=minutes;
mL=milliliters;
mmol=millimoles;
mol=moles;

### SEM=CH₂CH₂SiMe₃

### Bn=Benzyl

### DPM=Diphenylmethyl

PMB=p-methoxybenzyl
DMPM=3,4-dimethoxybenzyl

### Boc=COOC(CH₃)₃

### MOC=COOCH₃

### EOC=COOCH₂CH₃

### Alloc=COOAllyl

### Cbz and Z=COOBenzyl

### Moz and MeOZ=COO-p-methoxybenzyl

### Teoc=COOCH₂CH₂SiMe₃

### FMoc=9-Fluorenylmethyloxycarbonyl

### Troc=COOCH₂CH₂CCl₃

### Formyl=COH

### Ac=COMe

### Bz=COPh

### Trifluoroacetyl=COCF₃

Tosyl=—OSO₂tolyl
Besyl=—OSO₂phenyl

### Mesyl=—OSO₂Me

### Nf=—OSO₂C₄F₉

### Tf=—OSO₂CF₃

Xantphos=4,5-Bis(diphenylphosphino)-9,9-dimethylxanthene

In one aspect of the invention, there is provided a composition comprising a compound having the following formula:

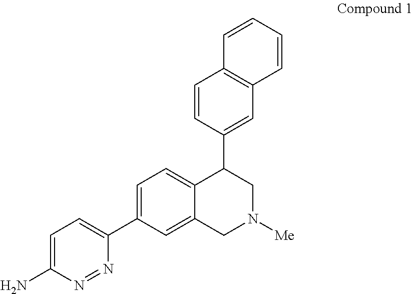 [SMILES: CN1Cc2cc(-c3ccc(N)nn3)ccc2C(c2ccc3ccccc3c2)C1]

and a compound having the following formula:

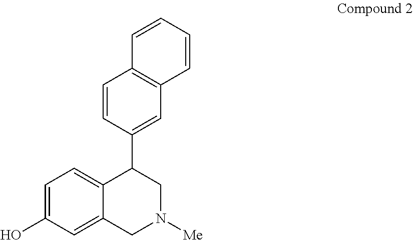 [SMILES: CN1Cc2cc(O)ccc2C(c2ccc3ccccc3c2)C1]

said Compound 2 present in an amount of from about 1 ppm to less than 1.0 wt % based on the weight of Compound 1. Preferably, the amount of Compound 2 is less than about 1500 ppm based on the weight of Compound 1. Preferably, the amount of Compound 2 is less than about 500 ppm based on the weight of Compound 1. Often, the lower level of Compound 2 may be about 10 ppm or 25 ppm, or higher, As such, the typical ranges for Compound 2, based on the weight of Compound 1, may be about 10 ppm to less than 1.0 wt %, about 10 ppm to less than about 1500 ppm, about 10 ppm to less than about 500 ppm, about 25 ppm to less than 1.0 wt %, about 25 ppm to less than about 1500 ppm, and about 25 ppm to less than about 500 ppm.

In another aspect of the invention, there is provided a composition comprising a compound having the following formula:

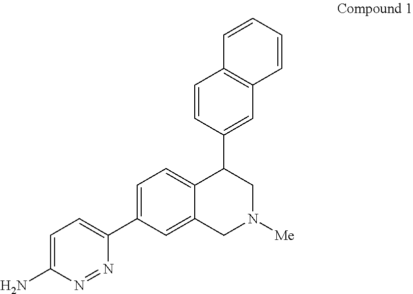 [SMILES: CN1Cc2cc(-c3ccc(N)nn3)ccc2C(c2ccc3ccccc3c2)C1]

and a compound having the following formula:

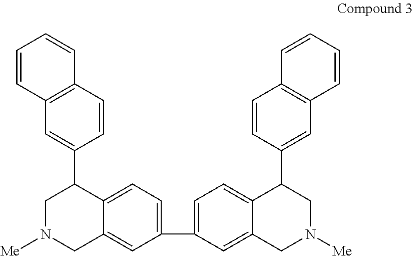 [SMILES: CN1Cc2cc(-c3ccc4c(c3)CN(C)CC4c3ccc4ccccc4c3)ccc2C(c2ccc3ccccc3c2)C1]

said Compound 3 present in an amount of from about 1 ppm to less than 1.0 wt % based on the weight of Compound 1. Preferably, the amount of Compound 3 is less than about 1500 ppm based on the weight of Compound 1. Preferably, the amount of Compound 3 is less than about 500 ppm based on the weight of Compound 1. Often, the lower level of Compound 3 may be about 10 ppm or 25 ppm, or higher, As such, the typical ranges for Compound 3, based on the weight of Compound 1, may be about 10 ppm to less than 1.0 wt %, about 10 ppm to less than about 1500 ppm, about 10 ppm to less than about 500 ppm, about 25 ppm to less than 1.0 wt %, about 25 ppm to less than about 1500 ppm, and about 25 ppm to less than about 500 ppm.

In another aspect of the invention, there is provided a composition of claim 1 further comprising a Compound 3 in an amount of from about 1 ppm to less than 1.0 wt % based on the weight of Compound 1.

In another aspect of the invention, there is provided a compound having the formula:

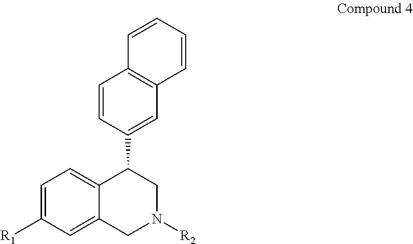

wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃, benzyl, diphenylmethyl, p-methoxybenzyl, 3,4-dimethoxybenzyl, COOC(CH₃)₃; COOCH₃, COOCH₂CH₃, COOAllyl, COObenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃, and
R₃ is selected from the group consisting of alkyl, aryl and heteroaryl;
and/or salts thereof.

In another aspect of the invention, there is provided a compound having the formula:

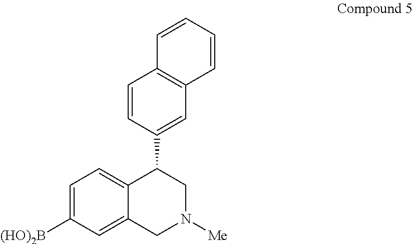 [SMILES: CN1Cc2cc(B(O)O)ccc2[C@H](c2ccc3ccccc3c2)C1]

complexed as a salt of di-p-toluoyl-D-tartaric acid.

In another aspect of the invention, there is provided a compound having the following formula:

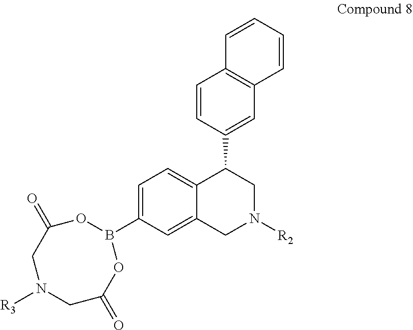

wherein
R₂ is selected from the group consisting of H, CH3, CH₂CH₂SiMe₃, Benzyl, Diphenylmethyl, p-methoxybenzyl, 3,4-dimethoxybenzyl, COOC(CH₃)₃; COOCH₃ (MOC), COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃; and
R₃ is selected from the group consisting of alkyl, aryl and heteroaryl; and/or salts thereof;

In another aspect of the invention, there is provided a compound having the following formula:

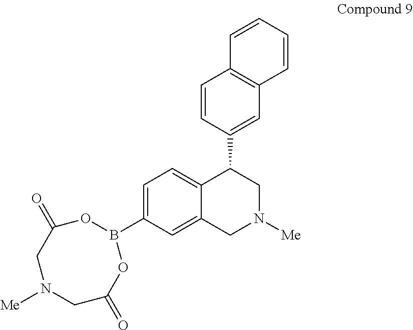 [SMILES: CN1CC(=O)OB(c2ccc3c(c2)CN(C)C[C@H]3c2ccc3ccccc3c2)OC(=O)C1]

complexed as a salt of di-p-toluoyl-D-tartaric acid.

In another aspect of the invention, there is provided a compound having the formula:

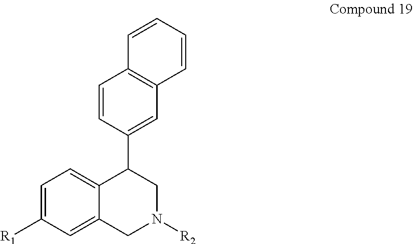

wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃, benzyl, diphenylmethyl, p-methoxybenzyl, 3,4-dimethoxybenzyl, COOC(CH₃)₃; COOCH₃, COOCH₂CH₃, COOAllyl, COObenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃, and
R₃ is selected from the group consisting of alkyl, aryl and heteroaryl;
and/or salts thereof
formula:

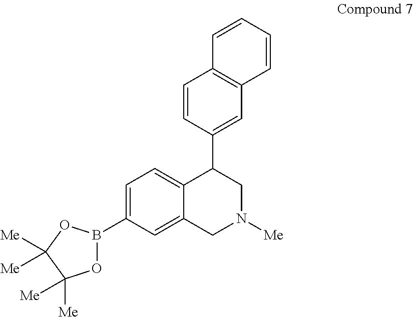 [SMILES: CN1Cc2cc(B3OC(C)(C)C(C)(C)O3)ccc2C(c2ccc3ccccc3c2)C1]

In another aspect of the invention, there is provided a compound having the the following formula:

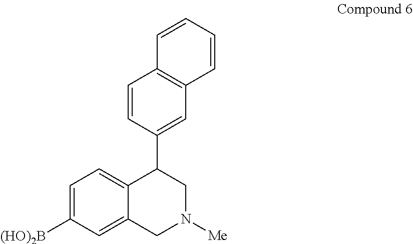 [SMILES: CN1Cc2cc(B(O)O)ccc2C(c2ccc3ccccc3c2)C1]

In another aspect of the invention, there is provided a compound having the following formula:

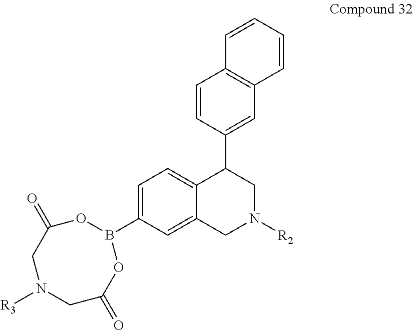

wherein
R₂ is selected from the group consisting of H, CH3, CH₂CH₂SiMe₃, Benzyl, Diphenylmethyl, p-methoxybenzyl, 3,4-dimethoxybenzyl, COOC(CH₃)₃; COOCH₃ (MOC), COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃; and
R₃ is selected from the group consisting of alkyl, aryl and heteroaryl;
and/or salts thereof;

In another aspect of the invention, there is provided a compound having the following formula:

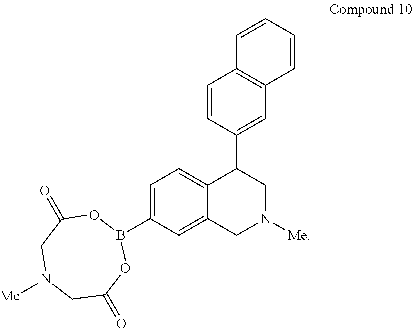 [SMILES: CN1CC(=O)OB(c2ccc3c(c2)CN(C)CC3c2ccc3ccccc3c2)OC(=O)C1]

In another aspect of the invention, there is provided a compound having the following formula:

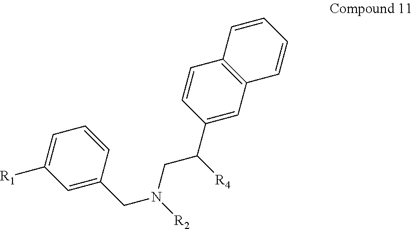

wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃, Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃,
R₃ is selected from the group consisting of alkyl, aryl and heteroaryl; and
R₄ is selected from the group consisting of —OH, —OMe, —OSO₂tolyl (Tosyl), —OSO₂phenyl (Besyl), —OSO₂Me (mesyl), —OSO₂C₄F₉(Nf), —OSO₂CF₃ (Tf), —OSO₂F, —Cl, —Br, —I, —OCHO, —O₂CMe (Ac), —O₂CPh (Bz), —ONO₂, R—OPO(OH)₂
and/or salts thereof;

In another aspect of the invention, there is provided a compound having the following formula:

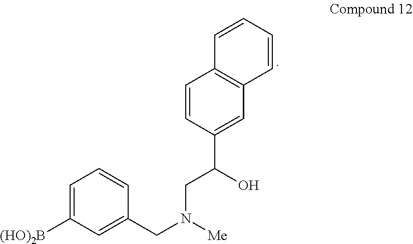 [SMILES: CN(Cc1cccc(B(O)O)c1)CC(O)c1ccc2ccccc2c1]

In another aspect of the invention, there is provided a compound having the formula:

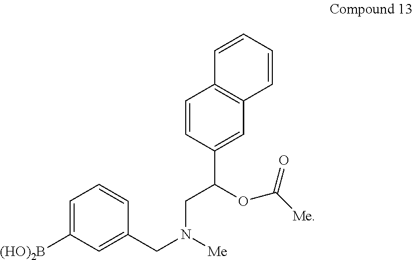 [SMILES: CC(=O)OC(CN(C)Cc1cccc(B(O)O)c1)c1ccc2ccccc2c1]

In another aspect of the invention, there is provided a compound having the formula:

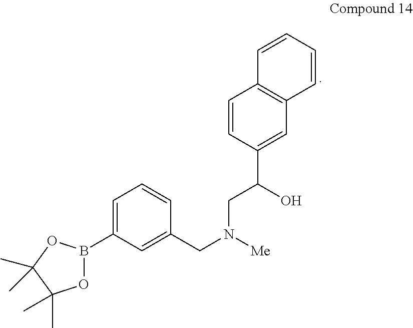 [SMILES: CN(Cc1cccc(B2OC(C)(C)C(C)(C)O2)c1)CC(O)c1ccc2ccccc2c1]

In another aspect of the invention, there is provided a compound having the following formula:

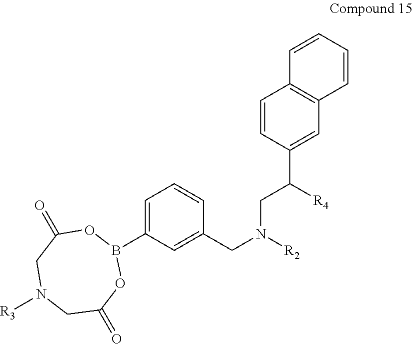

wherein
R₂ is selected from the group consisting of H, CH₃, CH₂CH₂SiMe₃, Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃;
R₃ is selected from the group consisting of alkyl, aryl and heteroaryl;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;

In another aspect of the invention, there is provided a compound the following formula:

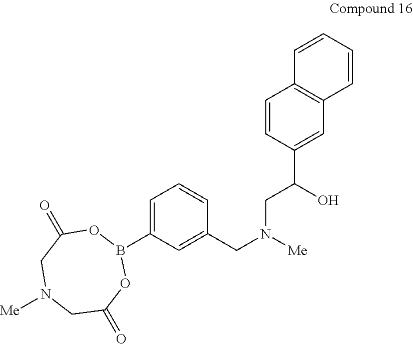 [SMILES: CN1CC(=O)OB(c2cccc(CN(C)CC(O)c3ccc4ccccc4c3)c2)OC(=O)C1]

In another aspect of the invention, there is provided a compound having the following formula:

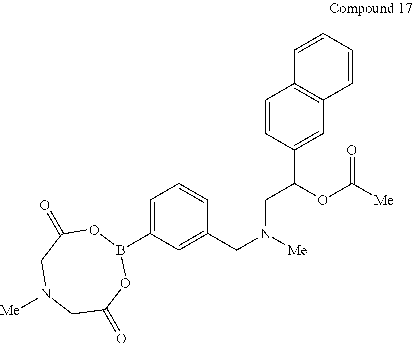 [SMILES: CC(=O)OC(CN(C)Cc1cccc(B2OC(=O)CN(C)CC(=O)O2)c1)c1ccc2ccccc2c1]

In another aspect of the invention, there is provided a compound having the formula:

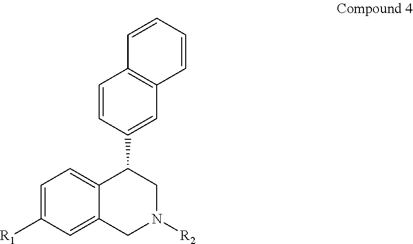

wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃, Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃,
and/or salts thereof
comprising reacting a compound having the formula:

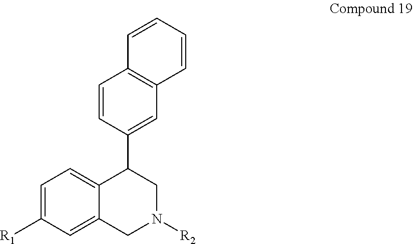

R₁ is selected from the group consisting of B(OH)₂, B(OR₃)₂, B(O₂COR₃)₂, and —BF₃K;
R₂ is selected from the group consisting of H, CH₃, CH₂CH₂SiMe₃, Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃,
and/or salts thereof
with a compound having the formula:

R₃—CO₂H

wherein
R₃ is selected from the group consisting of alkyl, aryl and heteroaryl;
under conditions effective to promote the formation of Compound 4.

In another aspect of the invention, there is provided a process of making a compound having the formula:

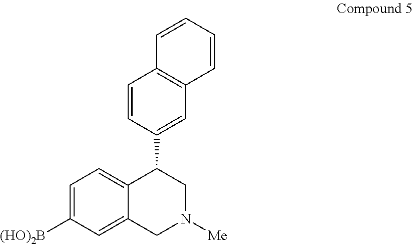 [SMILES: CN1Cc2cc(B(O)O)ccc2[C@H](c2ccc3ccccc3c2)C1]

complexed as a salt of di-p-toluoyl-D-tartaric acid
comprising reacting a compound having the formula:

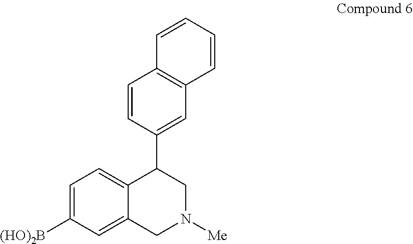 [SMILES: CN1Cc2cc(B(O)O)ccc2C(c2ccc3ccccc3c2)C1]

with a compound having the formula:

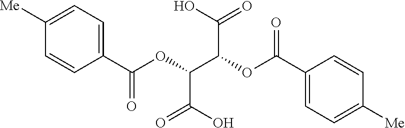 [SMILES: Cc1ccc(C(=O)O[C@@H](C(=O)O)[C@@H](OC(=O)c2ccc(C)cc2)C(=O)O)cc1]

di-p-toluoyl-D-tartaric acid
   under conditions effective to promote the formation of Compound 5.

In another aspect of the invention, there is provided a process of making a compound having the formula:

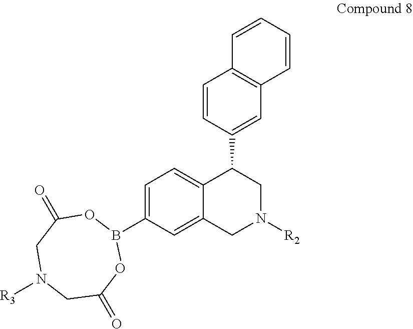

wherein; R₂ is selected from the group consisting of H, CH3, CH₂CH₂SiMe₃, Benzyl, Diphenylmethyl, p-methoxybenzyl, 3,4-dimethoxybenzyl, COOC(CH₃)₃; COOCH₃ (MOC), COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃; and
R₃ is selected from the group consisting of alkyl, aryl and heteroaryl; and/or salts thereof;
complexed as a salt of di-p-toluoyl-D-tartaric acid
comprising reacting a compound having the formula:

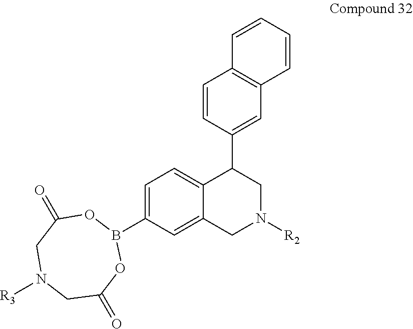

wherein R₂ is selected from the group consisting of H, CH3, CH₂CH₂SiMe₃, Benzyl, Diphenylmethyl, p-methoxybenzyl, 3,4-dimethoxybenzyl, COOC(CH₃)₃; COOCH₃ (MOC), COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃; and
R₃ is selected from the group consisting of alkyl, aryl and heteroaryl;
and/or salts thereof;
with a compound having the formula:

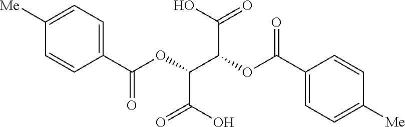 [SMILES: Cc1ccc(C(=O)O[C@@H](C(=O)O)[C@@H](OC(=O)c2ccc(C)cc2)C(=O)O)cc1]

di-p-toluoyl-D-tartaric acid
   under conditions effective to promote the formation of Compound 8.

In another aspect of the invention, there is provided a process of making a compound having the formula:

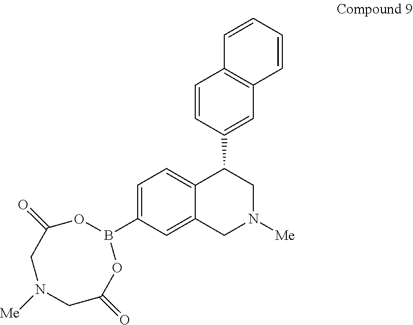 [SMILES: CN1CC(=O)OB(c2ccc3c(c2)CN(C)C[C@H]3c2ccc3ccccc3c2)OC(=O)C1]

complexed as a salt of di-p-toluoyl-D-tartaric acid
comprising reacting a compound having the formula:

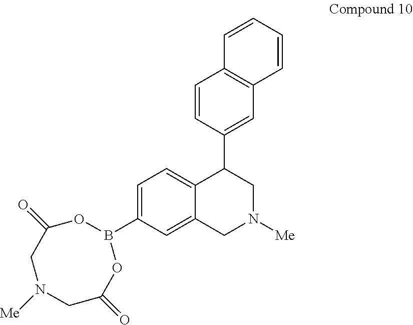 [SMILES: CN1CC(=O)OB(c2ccc3c(c2)CN(C)CC3c2ccc3ccccc3c2)OC(=O)C1]

with a compound having the formula:

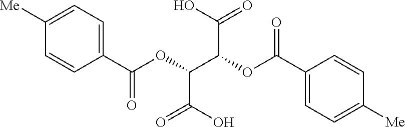 [SMILES: Cc1ccc(C(=O)O[C@@H](C(=O)O)[C@@H](OC(=O)c2ccc(C)cc2)C(=O)O)cc1]

di-p-toluoyl-D-tartaric acid
   under conditions effective to promote the formation of Compound 9.

In another aspect of the invention, there is provided a process of making a compound having the formula:

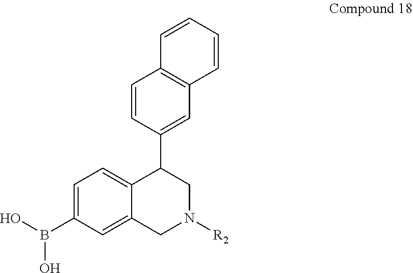

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH (formyl), COMe, COPh, and COCF₃;
comprising reacting a compound having the formula:

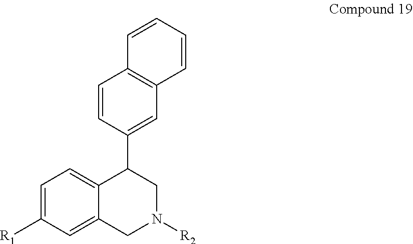

wherein
R₁ is selected from the group consisting of B(OR₃)₂, B(O₂COR₃)₂;
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃; R₃ is selected from the group consisting of alkyl, aryl and heteroaryl;
and/or salts thereof;
under conditions effective to promote the formation of Compound 18.

In another aspect of the invention, there is provided a process of making a compound having the formula:

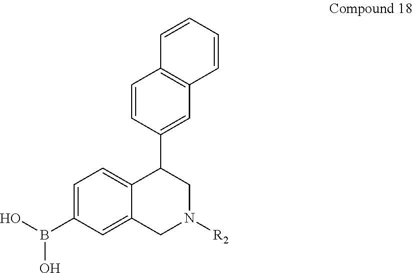

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH (formyl), COMe, COPh, and COCF₃;
comprising reacting a compound having the formula:

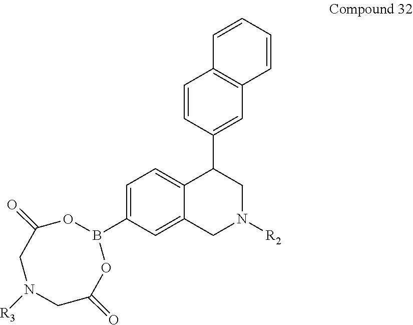

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃;
R₃ is selected from the group consisting of alkyl, aryl and heteroaryl;
and/or salts thereof;
under conditions effective to promote the formation of Compound 18.

In another aspect of the invention, there is provided a process of making a compound having the formula:

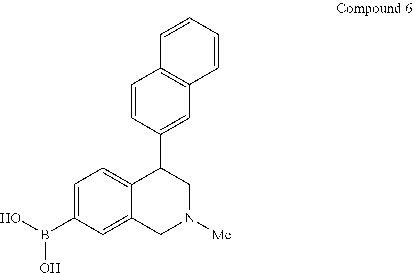 [SMILES: CN1Cc2cc(B(O)O)ccc2C(c2ccc3ccccc3c2)C1]

comprising reacting a compound having the formula:

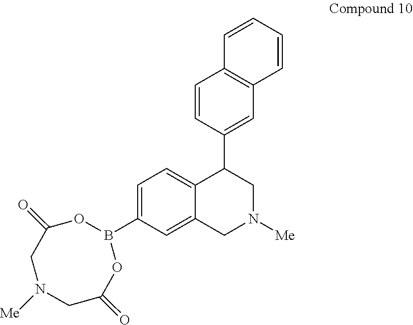 [SMILES: CN1CC(=O)OB(c2ccc3c(c2)CN(C)CC3c2ccc3ccccc3c2)OC(=O)C1]

under conditions effective to promote the formulation of Compound 6.

In another aspect of the invention, there is provided a process of making a compound having the formula:

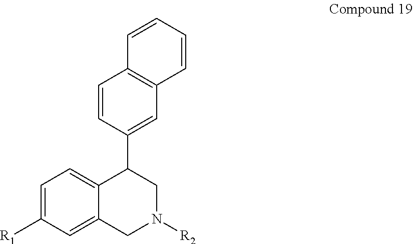

wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃
R₃ is selected from the group consisting of alkyl, aryl and heteroaryl;
and/or salts thereof;
comprising reacting a compound having the formula:

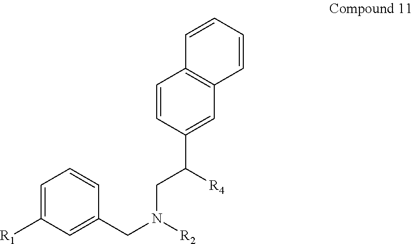

wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃;
R₃ is selected from the group consisting of alkyl, aryl and heteroaryl;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 19.

In another aspect of the invention, there is provided a process of making a compound having the formula:

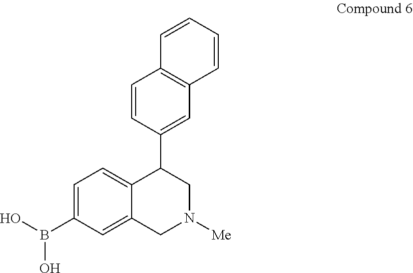 [SMILES: CN1Cc2cc(B(O)O)ccc2C(c2ccc3ccccc3c2)C1]

comprising reacting a compound having the formula:

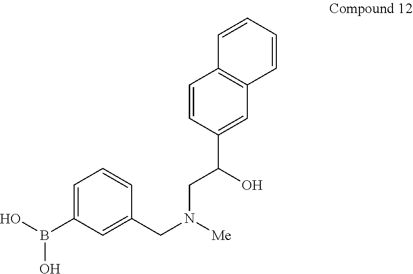 [SMILES: CN(Cc1cccc(B(O)O)c1)CC(O)c1ccc2ccccc2c1]

under conditions effective to promote the formation of Compound 6.

In another aspect of the invention, there is provided a process of making a compound having the formula:

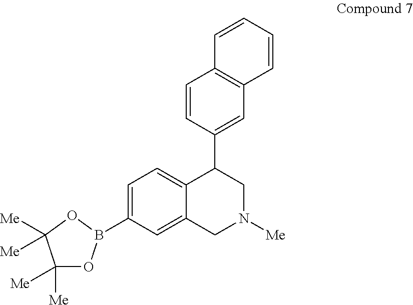 [SMILES: CN1Cc2cc(B3OC(C)(C)C(C)(C)O3)ccc2C(c2ccc3ccccc3c2)C1]

comprising reacting a compound having the formula:

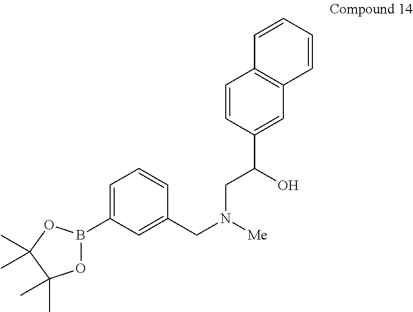 [SMILES: CN(Cc1cccc(B2OC(C)(C)C(C)(C)O2)c1)CC(O)c1ccc2ccccc2c1]

under conditions effective to promote the formation of Compound 7.

In another aspect of the invention, there is provided a process of making a compound having the formula:

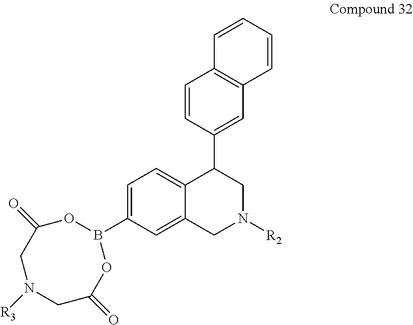

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃; and/or salts thereof;
comprising reacting a compound having the formula:

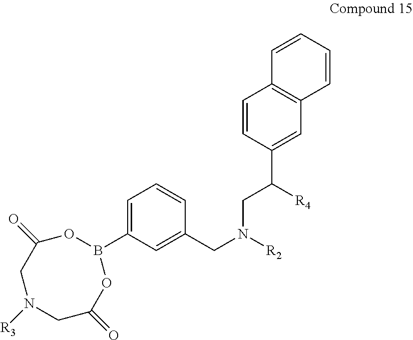

R₂ is selected from the group consisting of H, CH₃, CH₂CH₂SiMe₃, Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃;
R₃ is selected from the group consisting of alkyl, aryl and heteroaryl;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 32.

In another aspect of the invention, there is provided a process of making a compound having the formula:

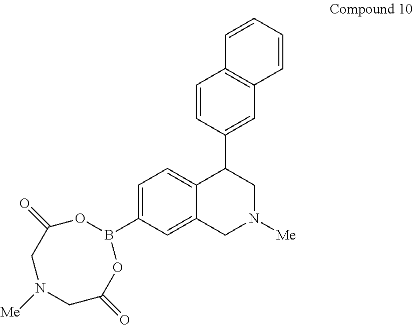 [SMILES: CN1CC(=O)OB(c2ccc3c(c2)CN(C)CC3c2ccc3ccccc3c2)OC(=O)C1]

comprising reacting a compound having the formula:

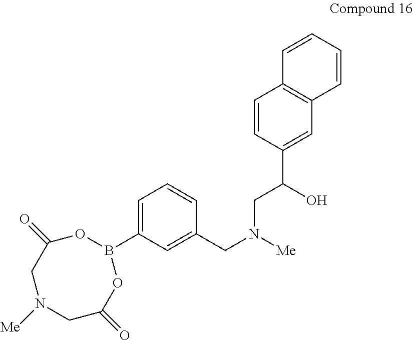 [SMILES: CN1CC(=O)OB(c2cccc(CN(C)CC(O)c3ccc4ccccc4c3)c2)OC(=O)C1]

under conditions effective to promote the formation of Compound 10.

In another aspect of the invention, there is provided a process of making a compound having the formula:

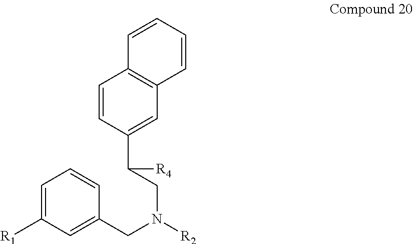

wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃;
R₃ is selected from the group consisting of alkyl, aryl and heteroaryl;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh

### Compound 21, —ONO₂, R—OPO(OH)₂

and/or salts thereof;
comprising reacting a compound having the formula:

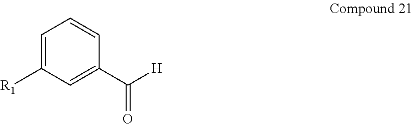

wherein
R₁ is selected from the group consisting of B(OH)₂, B(OR₃)₂, B(O₂COR₃)₂, and —BF₃K;
R₃ is selected from the group consisting of alkyl, aryl and heteroaryl;
and/or salts thereof;
with a compound having the formula:

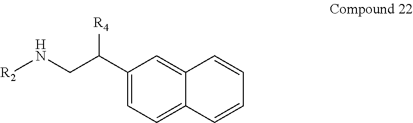

wherein
R₂ is selected from the group consisting of H, CH3, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃; R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 20.

In another aspect of the invention, there is provided a process of making a compound having the formula:

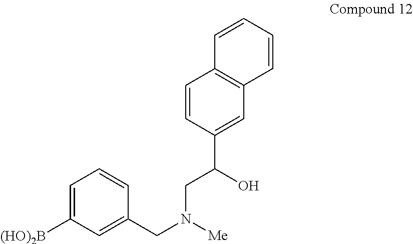 [SMILES: CN(Cc1cccc(B(O)O)c1)CC(O)c1ccc2ccccc2c1]

comprising reacting a compound having the formula:

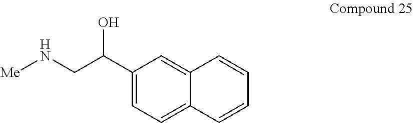 [SMILES: CNCC(O)c1ccc2ccccc2c1]

with a compound of the formula

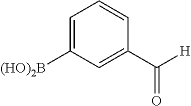 [SMILES: O=Cc1cccc(B(O)O)c1]

under conditions effective to promote the formation of Compound 12.

In another aspect of the invention, there is provided a process of making a compound having the formula:

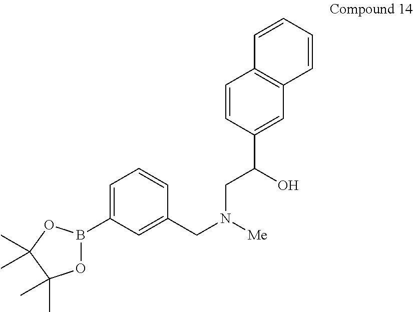 [SMILES: CN(Cc1cccc(B2OC(C)(C)C(C)(C)O2)c1)CC(O)c1ccc2ccccc2c1]

comprising reacting a compound having the formula:

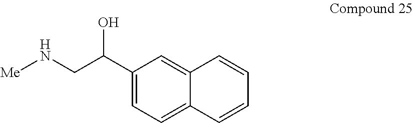 [SMILES: CNCC(O)c1ccc2ccccc2c1]

with a compound of the formula

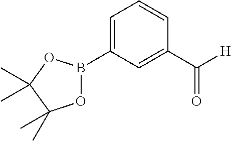 [SMILES: CC1(C)OB(c2cccc(C=O)c2)OC1(C)C]

under conditions effective to promote the formation of Compound 14.

In another aspect of the invention, there is provided a process of making a compound having the formula:

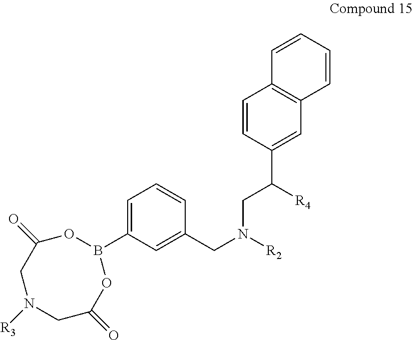

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃;
R₃ is selected from the group consisting of alkyl, aryl and heteroaryl;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh Compound 21, —ONO₂, R—OPO(OH)₂
and/or salts thereof;
comprising reacting a compound having the formula:

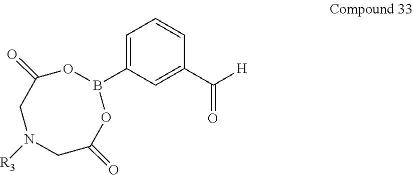

wherein
R₃ is selected from the group consisting of alkyl, aryl and heteroaryl;
and/or salts thereof;
with a compound having the formula:

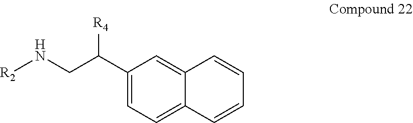

wherein
R₂ is selected from the group consisting of H, CH3, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃; R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 15.

In another aspect of the invention, there is provided a process of making a compound having the formula:

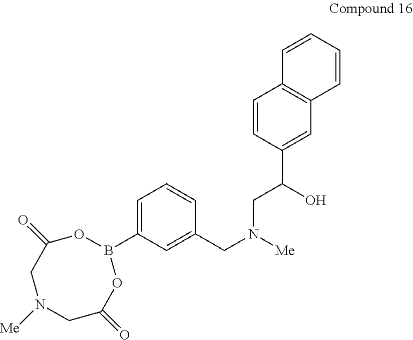 [SMILES: CN1CC(=O)OB(c2cccc(CN(C)CC(O)c3ccc4ccccc4c3)c2)OC(=O)C1]

comprising reacting a compound having the formula:

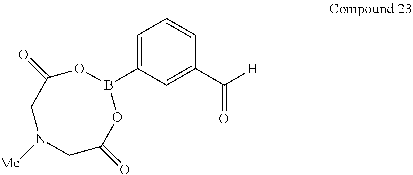 [SMILES: CN1CC(=O)OB(c2cccc(C=O)c2)OC(=O)C1]

with a compound of the formula

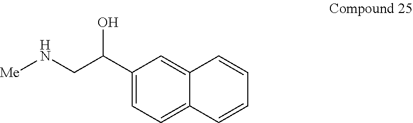 [SMILES: CNCC(O)c1ccc2ccccc2c1]

under conditions effective to promote the formation of Compound 16.

In another aspect of the invention, there is provided a process of making a compound having the formula:

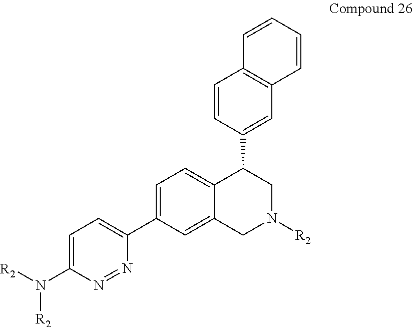

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃; and/or salts thereof; wherein each said R₂ may be the same or different;
comprising reacting a compound having the formula:

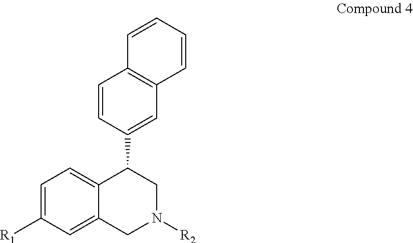

wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃ (Boc); COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃;
R₃ is selected from the group consisting of alkyl, aryl and heteroaryl;
and/or salts thereof;
with a compound having the formula:

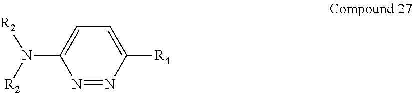

wherein
R₂ is selected from the group consisting of H, CH3, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃, wherein each said R₂ may be the same or different;
R₄ is selected from the group consisting of —OH, —OMe, —OSO₂tolyl, —OSO₂phenyl OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 26.

In another aspect of the invention, there is provided a process of making a compound having the formula:

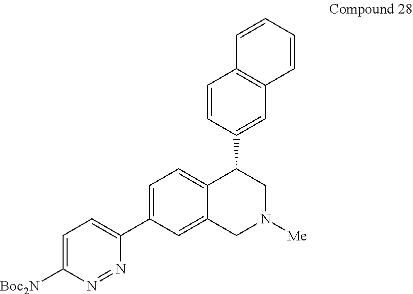

comprising reacting a compound having the formula:

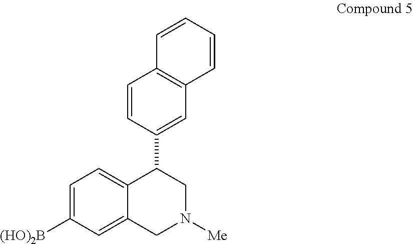

complexed as a salt of di-p-toluoyl-D-tartaric acid
with a compound having the formula:

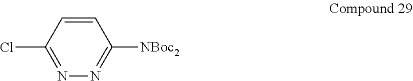

under conditions effective to promote the formation of Compound 28.

In another aspect of the invention, there is provided a process of making a compound having the formula:

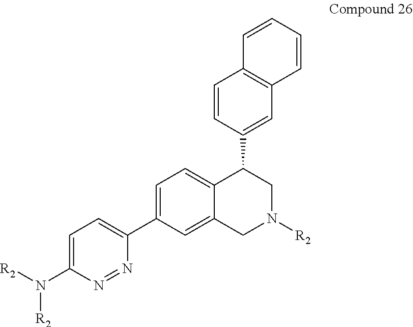

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃;
wherein each said R₂ may be the same or different
and/or salts thereof;
comprising reacting a compound having the formula:

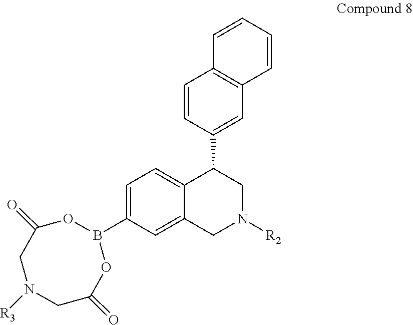

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃ (Boc); COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃; R₃ is selected from the group consisting of alkyl, aryl and heteroaryl;
and/or salts thereof;
with a compound having the formula:

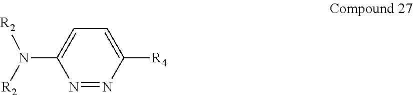

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, COCF₃, wherein each said R₂ may be the same or different;
R₄ is selected from the group consisting of —OH, —OMe, —OSO₂tolyl, —OSO₂phenyl OSO₂Me, —OSO₂C₄F₉, —OSO₂CF₃, —OSO₂F, —Cl, —Br, —I, —OCHO, —O₂CMe, —O₂CPh, —

### ONO₂, R—OPO(OH)₂

and/or salts thereof;
under conditions effective to promote the formation of Compound 26.

In another aspect of the invention, there is provided a process of making a compound having the formula:

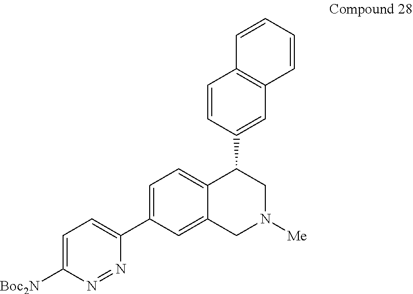

comprising reacting a compound having the formula:

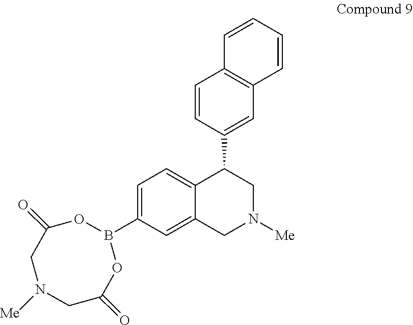 [SMILES: CN1CC(=O)OB(c2ccc3c(c2)CN(C)C[C@H]3c2ccc3ccccc3c2)OC(=O)C1]

complexed as a salt of di-p-toluoyl-D-tartaric acid
with a compound having the formula:

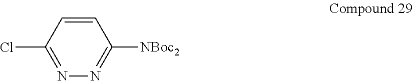

under conditions effective to promote the formation of Compound 28.

In another aspect of the invention, there is provided a process of making a compound having the formula:

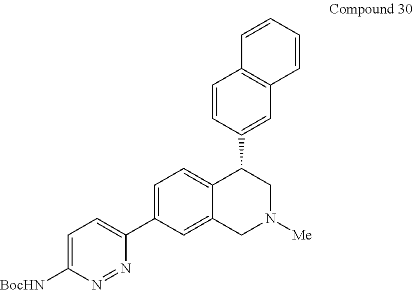 [SMILES: CN1Cc2cc(-c3ccc(NC(=O)OC(C)(C)C)nn3)ccc2[C@H](c2ccc3ccccc3c2)C1]

comprising reacting a compound having the formula:

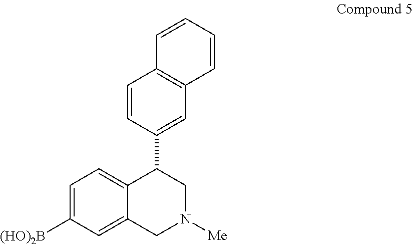

complexed as a salt of di-p-toluoyl-D-tartaric acid
with a compound having the formula:

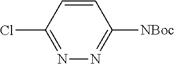

under conditions effective to promote the formation of Compound 30.

In another aspect of the invention, there is provided a process of making a compound having the formula:

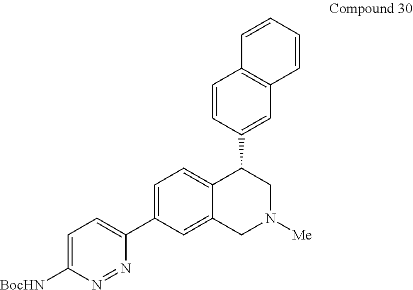 [SMILES: CN1Cc2cc(-c3ccc(NC(=O)OC(C)(C)C)nn3)ccc2[C@H](c2ccc3ccccc3c2)C1]

comprising reacting a compound having the formula:

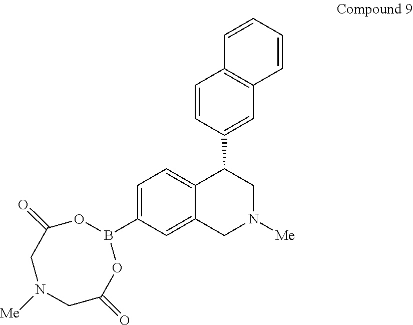 [SMILES: CN1CC(=O)OB(c2ccc3c(c2)CN(C)C[C@H]3c2ccc3ccccc3c2)OC(=O)C1]

complexed as a salt of di-p-toluoyl-D-tartaric acid
with a compound having the formula:

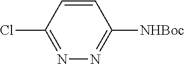 [SMILES: CC(C)(C)OC(=O)Nc1ccc(Cl)nn1]

under conditions effective to promote the formation of Compound 30.

In another aspect of the invention, there is provided a process of making a compound having the formula:

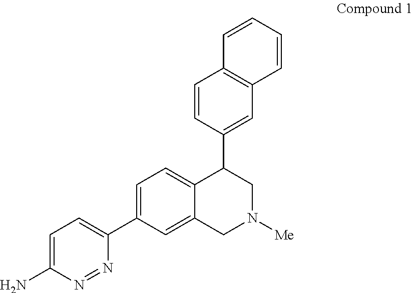 [SMILES: CN1Cc2cc(-c3ccc(N)nn3)ccc2C(c2ccc3ccccc3c2)C1]

comprising reacting a compound having the formula:

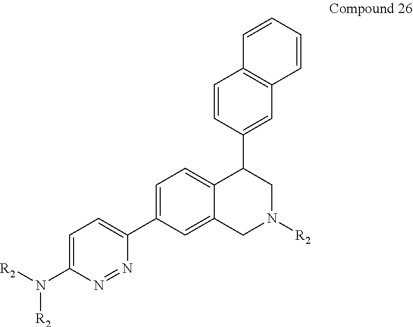

wherein
R₂ is selected from the group consisting of H, CH₃, CH₂CH₂SiMe₃; Benzyl, Diphenylmethyl, p-methoxybenzyl, 3,4-dimethoxybenzyl, COOC(CH₃)₃; COOCH₃, COOCH₂CH₃, COOAllyl, COOBenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃; wherein each said R₂ may be the same or different;
and/or salts thereof;
under conditions effective to promote the formation of Compound 1.

In another aspect of the invention, there is provided a process of making a compound having the formula:

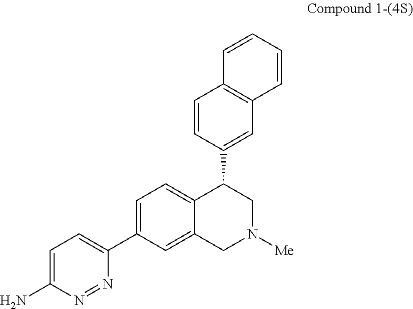 [SMILES: CN1Cc2cc(-c3ccc(N)nn3)ccc2[C@H](c2ccc3ccccc3c2)C1]

comprising reacting a compound having the formula:

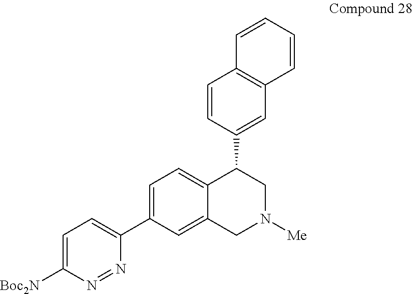

under conditions effective to promote the formation of Compound 1-(4S).

In another aspect of the invention, there is provided a process of making a compound having the formula:

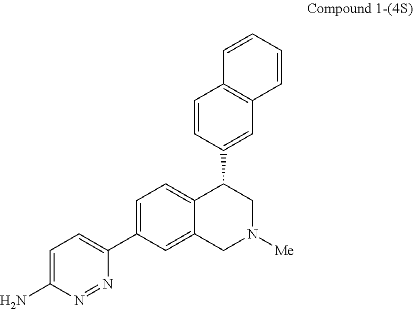 [SMILES: CN1Cc2cc(-c3ccc(N)nn3)ccc2[C@H](c2ccc3ccccc3c2)C1]

comprising reacting a compound having the formula:

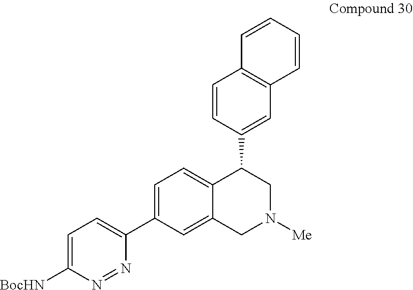 [SMILES: CN1Cc2cc(-c3ccc(NC(=O)OC(C)(C)C)nn3)ccc2[C@H](c2ccc3ccccc3c2)C1]

under conditions effective to promote the formation of Compound 1-(4S).

In another aspect of the invention, there is provided a process of making a compound having the formula:

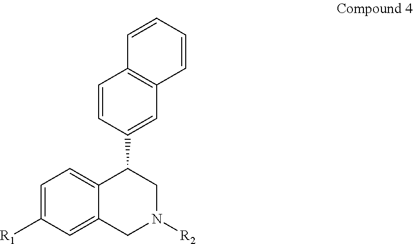

wherein
R₁ is selected from the group consisting of B(OH)₂, B(OR₃)₂, B(O₂COR₃)₂, and —BF₃K;
R₂ is a protecting group;
R₃ is selected from the group consisting of alkyl, aryl and heteroaryl;
comprising reacting a compound having the formula:

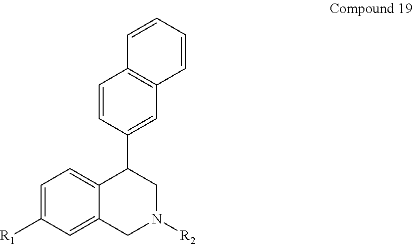

R₁ is selected from the group consisting of B(OH)₂, B(OR₃)₂, B(O₂COR₃)₂, and —BF₃K;
R₂ is said protecting group;
R₃ is selected from the group consisting of alkyl, aryl and heteroaryl;
with a compound having the formula:

R₃—CO₂H

wherein
R₃ is selected from the group consisting of alkyl, aryl and heteroaryl;
under conditions effective to promote the formation of Compound 4.

In another aspect of the invention, there is provided a process of making a compound having the formula:

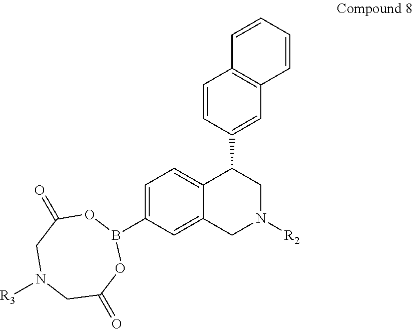

wherein R₂ is a protecting group;
R₃ is selected from the group consisting of alkyl, aryl and heteroaryl;
complexed as a salt of di-p-toluoyl-D-tartaric acid
comprising reacting a compound having the formula:

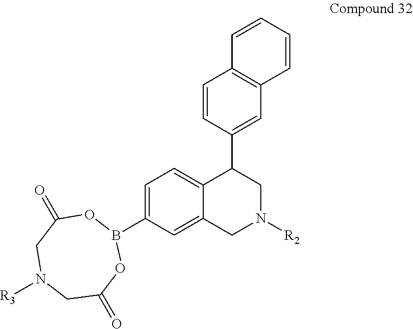

wherein R₂ is said protecting group; and
R₃ is selected from the group consisting of alkyl, aryl and heteroaryl;
and/or salts thereof;
with a compound having the formula:

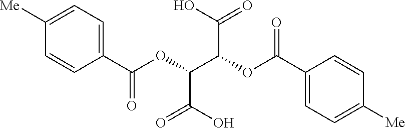 [SMILES: Cc1ccc(C(=O)O[C@@H](C(=O)O)[C@@H](OC(=O)c2ccc(C)cc2)C(=O)O)cc1]

di-p-toluoyl-D-tartaric acid
   under conditions effective to promote the formation of Compound 8.

In another aspect of the invention, there is provided a process of making a compound having the formula:

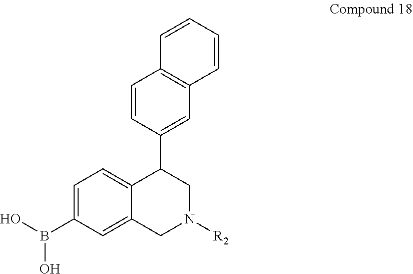

wherein
R₂ is a protecting group;
comprising reacting a compound having the formula:

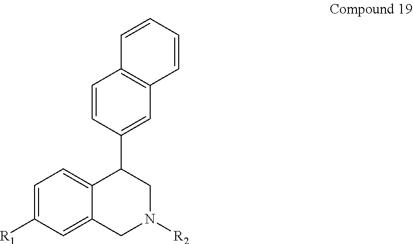

wherein
R₁ is selected from the group consisting of B(OR₃)₂, B(O₂COR₃)₂;
R₂ is said protecting group;
R₃ is selected from the group consisting of alkyl, aryl and heteroaryl;
and/or salts thereof;
under conditions effective to promote the formation of Compound 18.

In another aspect of the invention, there is provided a process of making a compound having the formula:

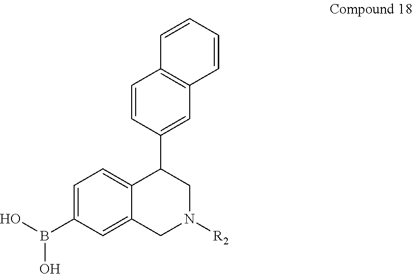

wherein
R₂ is a protecting group;
comprising reacting a compound having the formula:

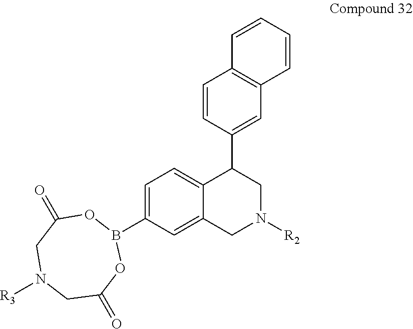

wherein
R₂ is said protecting group;
R₃ is selected from the group consisting of alkyl, aryl and heteroaryl;
under conditions effective to promote the formation of Compound 18.

In another aspect of the invention, there is provided a process of making a compound having the formula:

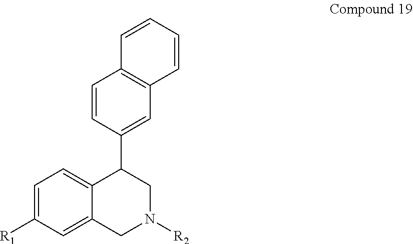

wherein
R₁ is selected from the group consisting of B(OH)₂, B(OR₃)₂, B(O₂COR₃)₂, and —BF₃K;
R₂ is a protecting group;
R₃ is selected from the group consisting of alkyl, aryl and heteroaryl;
and/or salts thereof;
comprising reacting a compound having the formula:

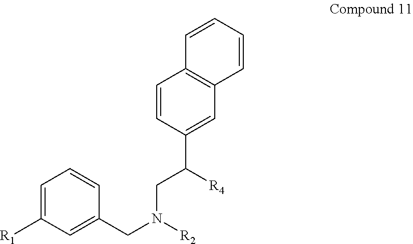

wherein
R₁ is selected from the group consisting of B(OH)₂, B(OR₃)₂, B(O₂COR₃)₂, and —BF₃K;
R₂ is said protecting group;
R₃ is selected from the group consisting of alkyl, aryl and heteroaryl;
under conditions effective to promote the formation of Compound 19.

In another aspect of the invention, there is provided a process of making a compound having the formula:

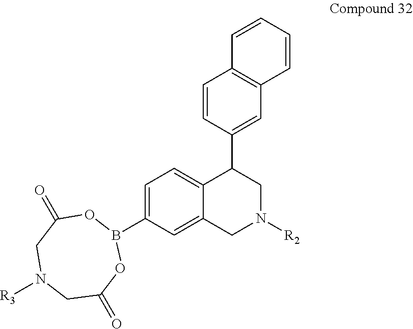

wherein
R₂ is a protecting group;
R₃ is selected from the group consisting of alkyl, aryl and heteroaryl;
and/or salts thereof;
comprising reacting a compound having the formula:

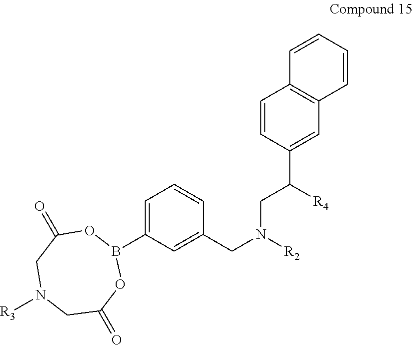

R₂ is said protecting group;
R₃ is selected from the group consisting of alkyl, aryl and heteroaryl;
R₄ is selected from the group consisting of —OH, —OMe, —OSO₂tolyl, —OSO₂phenyl, —
OSO₂Me, —OSO₂C₄F₉, —OSO₂CF₃, —OSO₂F, —Cl, —Br, —I, —OCHO, —O₂CMe, —O₂CPh, —

### ONO₂, R—OPO(OH)₂

and/or salts thereof;
under conditions effective to promote the formation of Compound 32.

In another aspect of the invention, there is provided a process of making a compound having the formula:

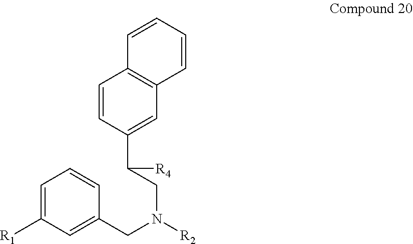

wherein
R₁ is selected from the group consisting of B(OH)₂, B(OR₃)₂, B(O₂COR₃)₂, and —BF₃K;
R₂ is a protecting group;
R₃ is selected from the group consisting of alkyl, aryl and heteroaryl; R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh Compound 21, —ONO₂, R—OPO(OH)₂
and/or salts thereof;
comprising reacting a compound having the formula:

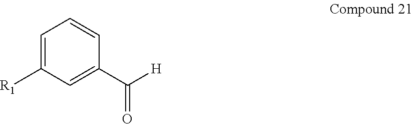

wherein
R₁ is selected from the group consisting of B(OH)₂, B(OR₃)₂, B(O₂COR₃)₂, and —BF₃K;
R₃ is selected from the group consisting of alkyl, aryl and heteroaryl;
and/or salts thereof;
with a compound having the formula:

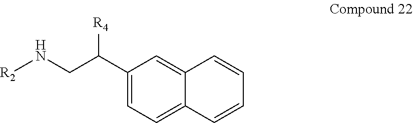

wherein
R₂ is said protecting group;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 20.

In another aspect of the invention, there is provided a process of making a compound having the formula:

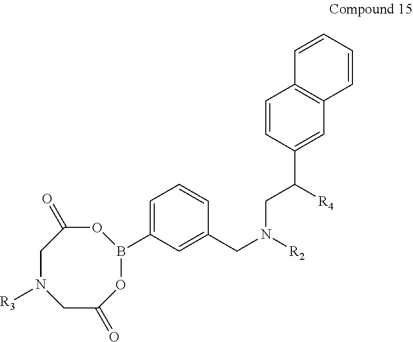

wherein
R₂ is a protecting group;
R₃ is selected from the group consisting of alkyl, aryl and heteroaryl;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh Compound 21, —ONO₂, R—OPO(OH)₂
and/or salts thereof;
comprising reacting a compound having the formula:

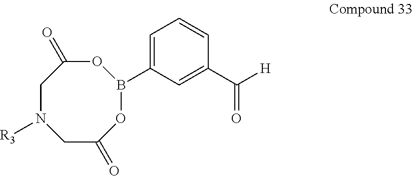

wherein
R₃ is selected from the group consisting of alkyl, aryl and heteroaryl;
and/or salts thereof;
with a compound having the formula:

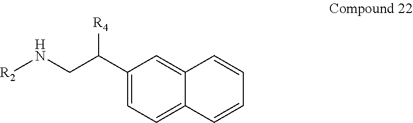

wherein
R₂ is said protecting group;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 15.

In another aspect of the invention, there is provided a process of making a compound having the formula:

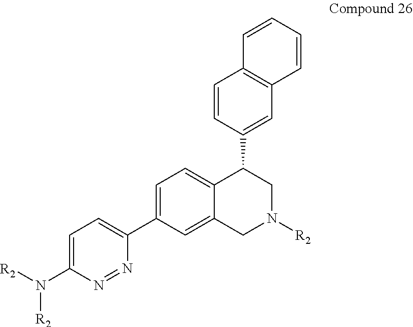

wherein
R₂ is a protecting group;
and/or salts thereof; wherein each said R₂ may be the same or different;
comprising reacting a compound having the formula:

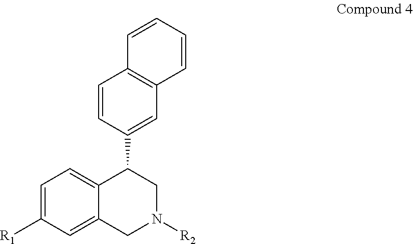

wherein
R₁ is selected from the group consisting of B(OH)₂, B(OR₃)₂, B(O₂COR₃)₂, and —BF₃K;
R₂ is said protecting group;
R₃ is selected from the group consisting of alkyl, aryl and heteroaryl;
and/or salts thereof;
with a compound having the formula:

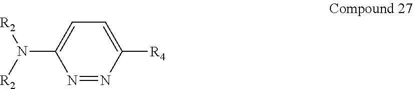

wherein
R₂ is said protecting group;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 26.

In another aspect of the invention, there is provided a process of making a compound having the formula:

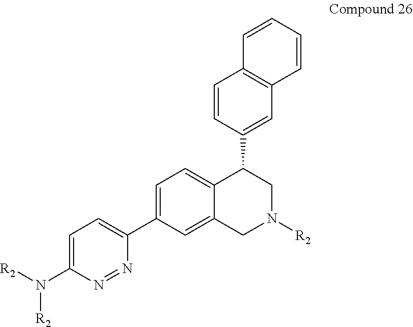

wherein
R₂ is a protecting group; wherein each said R₂ may be the same or different and/or salts thereof;
comprising reacting a compound having the formula:

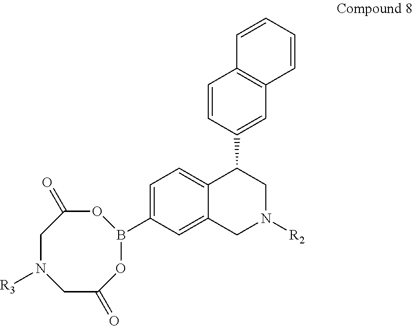

wherein
R₂ is said protecting group;
R₃ is selected from the group consisting of alkyl, aryl and heteroaryl;
and/or salts thereof;
with a compound having the formula:

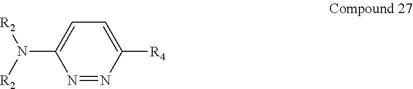

wherein
R₂ is said protecting group;
R₄ is selected from the group consisting of —OH, —OMe, —OSO₂tolyl, —OSO₂phenyl, —OSO₂Me, —OSO₂C₄F₉, —OSO₂CF₃, —OSO₂F, —Cl, —Br, —I, —OCHO, —O₂CMe, —O₂CPh, —ONO₂, R—OPO(OH)₂
and/or salts thereof;
under conditions effective to promote the formation of Compound 26.

In another aspect of the invention, there is provided a process of making a compound having the formula:

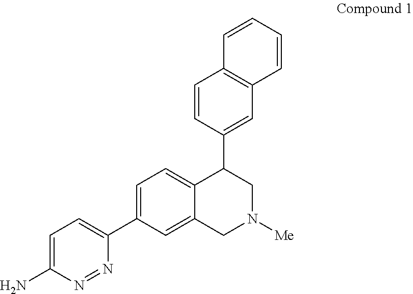 [SMILES: CN1Cc2cc(-c3ccc(N)nn3)ccc2C(c2ccc3ccccc3c2)C1]

comprising reacting a compound having the formula:

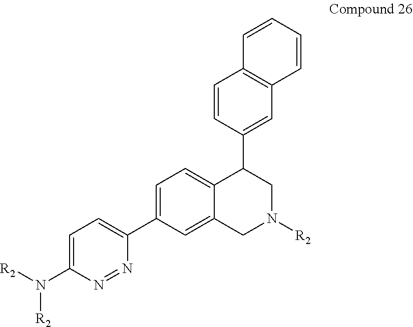

wherein
R₂ is a protecting group; wherein each said R₂ may be the same or different;
and/or salts thereof;
under conditions effective to promote the formation of Compound 1.

In another aspect of the invention, there is provided a composition comprising Compound 1 wherein Compound 1 is in the form of Compound 1-(4S). In another aspect of the invention, there is provided a composition comprising Compound 1 wherein Compound 1-(4S) is in the crystalline Form 1.

In another aspect of the invention, there is provided a composition comprising Compound 1-(4S) in the form of a tablet or capsule comprising from about 0.10 to 2.0 mg of Compound 1-(4S).

In another aspect of the invention, there is provided a composition comprising Compound 1-(4S) in the form of a tablet or capsule comprising from about 0.10 to 2.0 mg of Compound 1-(4S) further comprising a pharmaceutically acceptable additive.

In another aspect of the invention, there is provided a composition comprising Compound 1-(4S) in the form of a tablet or capsule comprising from about 0.10 to 2.0 mg of Compound 1-(4S) further comprising a pharmaceutically acceptable additive, the composition providing a total blood plasma concentration profile of Compound 1-(4S), as measured by AUC at 24 hours after an initial dose of the composition, that is at least greater than about 50% of the total blood plasma concentration as measured by AUC at 24 hours of an initial dose of an orally administered solution comprising Compound 1-(4S).

In another aspect of the invention, there is provided a composition comprising Compound 1-(4S) in the form of a tablet or capsule comprising from about 0.10 to 2.0 mg of Compound 1-(4S) further comprising a pharmaceutically acceptable additive, the composition providing a blood plasma concentration profile after an initial dose of the composition with a Cmax of Compound 1-(4S) after an initial dose of the composition that is at least greater than about 40% of the Cmax of an orally administered solution comprising Compound 1-(4S).

In another aspect of the invention, there is provided a method of treating depression comprising administering to a patient in need thereof a composition comprising Compound 1, preferably Compound 1-(4S).

In another aspect of the invention, there is provided a method of treating major depressive episode in an adult patient with major depressive disorder who has experienced inadequate response to separate trials of adequate dose and duration of two antidepressants from different classes in the current episode comprising administering to the patient a composition comprising Compound 1-(4S).

In another aspect of the invention, there is provided a method of treating depression comprising administering to a patient in need thereof a composition comprising Compound 1-(4S) wherein a total blood plasma concentration profile of Compound 1-(4S) is provided, as measured by AUC at 24 hours after an initial dose of the composition, that is at least greater than about 50% of the total blood plasma concentration as measured by AUC at 24 hours of an initial dose of an orally administered solution comprising Compound 1-(4S).

In another aspect of the invention, there is provided a method of treating depression comprising administering to a patient in need thereof a composition comprising Compound 1-(4S) wherein a blood plasma concentration profile after an initial dose of the composition has a Cmax of Compound 1-(4S) that is at least greater than about 40% of the Cmax of an orally administered solution comprising Compound 1-(4S).

The following reaction schemes are provided to illustrate the invention, but are not intended to limit that invention described herein. The reaction schemes are described, for example, with respect to the preparation of Compound 1, i.e.,

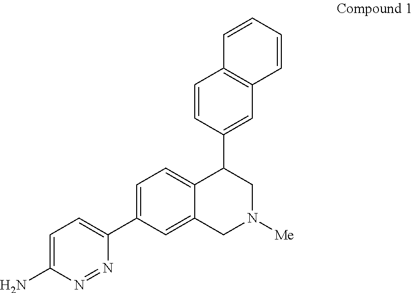 [SMILES: CN1Cc2cc(-c3ccc(N)nn3)ccc2C(c2ccc3ccccc3c2)C1]

Exemplary reagents and procedures for these reactions appear hereinafter or are described above. Starting materials are commercially available or can be readily prepared by one of ordinary skill in the art. The conditions effective to promote the formation of the desired compounds, e.g., solvents, reagents, acids, salts, protecting groups, techniques, catalysts, pressures, temperatures, reaction times, and the like, can be readily determined those skilled in the art. Representative synthesis schemes are illustrated below.

One preferred synthetic route can be represented as follows:

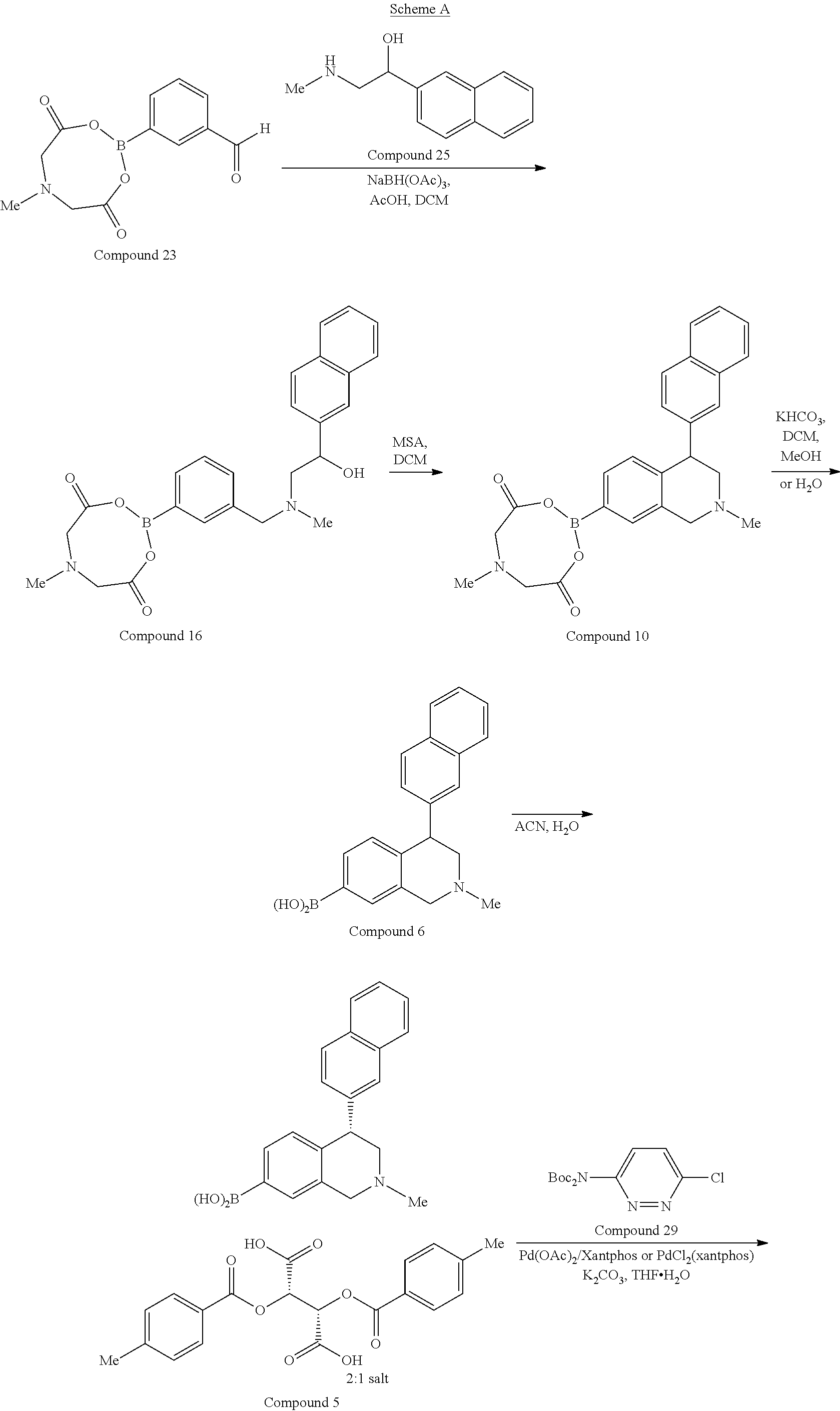

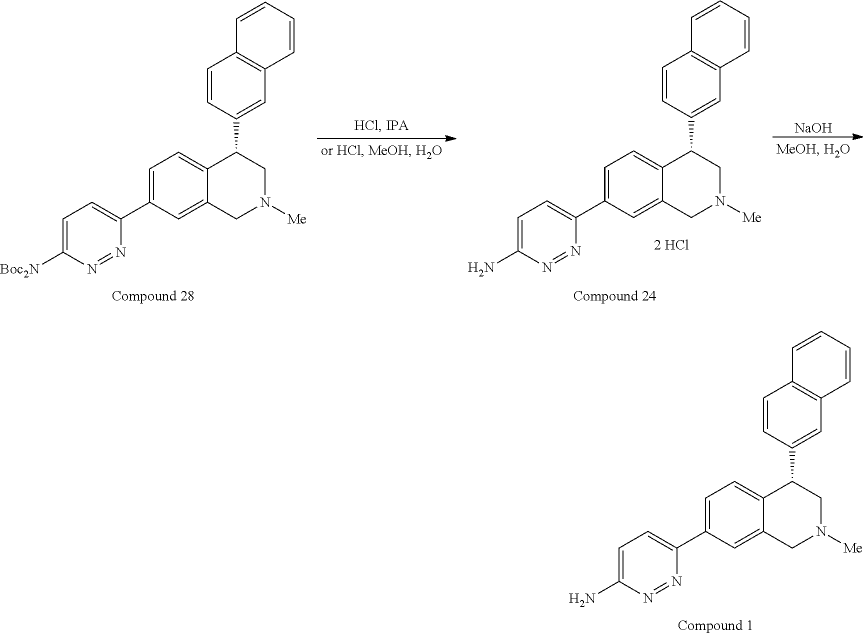

### Preparation of Compound 1

The synthesis of Compound 1-(4S) can utilize a general approach for C-7 and C-4 substituted tetrahydroisoquinolines. A functional handle at C-7, allows for the installation of the heterocycle using a palladium-catalyzed Suzuki-Miyaura cross-coupling reaction and the aryl substituent at C-4 is installed from an intermediate which can be readily prepared from commercial sources.

A preferred synthesis begins with the reductive amination of commercially available 3-(6-methyl-4,8-dioxo-1,3,6,2-dioxazaborocan-2-yl)benzaldehyde (Compound 23) and 2-(methylamino)-1-(naphthalen-2-yl)ethanol (Compound 25) using sodium triacetoxyborohydride in an appropriates solvent mixture such as DCM and AcOH. Alternative reagents and solvents can be used for this transformation including lewis acids and silanes, and hydrogenation conditions. The reductive amination yields the cyclization precursor (Compound 16) which is not isolated but used directly in the Friedel-Crafts cyclization by addition of MSA to afford the racemic tetrahydroisoquinoline core Compound 10 and its regioisomer in approximately 10:1 ratio. Alternative solvents, such as nitromethane, and acids can be used including Eaton's reagent, H₂SO₄ and several Lewis acids such as FeCl₃, InCl₃, TiCl₄, ZrCl₄. The reaction mixture is then directly hydrolyzed under either basic or acidic conditions leading to the formation of the corresponding boronic acid derivative Compound 6 and its regioisomer in approximately 10:1 ratio. The overall yield from Compound 23 is 65%. This mixture of regioisomers is then reacted with di-p-toluoyl-D-tartaric acid which allows for the classical resolution of the desired enantiomer which is isolated as 2:1 salt as as 95:5 mixture of diastereomers in 30-35% yield. The undesired regioisomer of Compound 6 is purged in the crystallization. The pyridazine ring is then installed via the palladium-catalyzed cross-coupling of Compounds 6 and 29, using palladium (II) precursors, xantphos as a ligand, and potassium carbonate as a base in a mixture of THF and water. Compound 28 is crystallized from the reaction mixture in optically pure form in 80-85% yield. Other ligands, bases and solvent systems can be used for the reaction. Compound 29 is readily prepared from commercially available 3-amino-6-chloropyridazine, Boc₂O, and DMAP in DMF. The Boc protecting groups are removed under acidic conditions using HCl in an alcoholic solvent such as IPA or MeOH to afford the di-HCl salt Compound 24. The free base, Compound 1, is isolated in 92-98% yield by crystallization from a mixture of aqueous sodium hydroxide and methanol.

The identification and quantification of Compound 1, Compound 2 and Compound 3 in the compositions of the present invention, as well as the other compounds and ingredients disclosed herein, can be readily determined by those skilled in the art, e.g., high pressure liquid chromatography (HPLC), mass spectroscopy (see, for example, Stroh J G, Petucci C J, Brecker S J, Nogle L M.

Sub-2 microm HPLC coupled with sub-ppm mass accuracy for analysis of pharmaceutical compound libraries, J Sep Sci. 2008 Dec. 31(21):3698-703.

In a preferred aspect, Compound 1-(4S) has the properties set for the in Table 5, below.

TABLE 5
Physical and Chemical Properties

| | |
|---|---|
| Chemical name | 6-[(4S)-1,2,3,4-tetrahydro-2-methyl-4-(2-naphthalenyl)- |
| of Compound | 7-isoquinolinyl]-3-pyridazinamine |
| 1-(4S) | |
| Chemical structure | 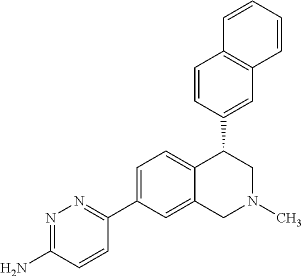 [SMILES: CN1Cc2cc(-c3ccc(N)nn3)ccc2[C@H](c2ccc3ccccc3c2)C1] |
| Molecular formula | C₂₄H₂₂N₄ |
| Molecular weight | 366.46 |
| Appearance | White to off-white powder |
| Melting point/ | 237°-243° C. |
| range | |
| Solution pH | ~7.0 at about 1 μg/mL concentration in water |
| pH-Solubility | At solution pH 6.5 and above, the aqueous solubility of |
| profile | Compound 1-(4S) is ~1 μg or less. Aqueous solubility |
| | increases at lower pH values (15.6 mg/mL at pH 2.0 |
| | and 1.76 mg/mL at pH 4.4) |
| Solubility profile | practically insoluble: n-heptane |
| (USP definition) | very slightly soluble: acetonitrile, ethyl acetate, |
| | n-butanol |
| | slightly soluble: isopropanol, acetone, ethanol, |
| | methanol, propylene glycol, dichloromethane |
| | sparingly soluble: PEG 400 |
| | soluble: N,N-dimethylacetamide, dimethylsulfoxide |
| | freely soluble: tetrahydrofuran |
| pKₐ | 4.9 and 7.8 |
| Distribution | Log D_{o/b} = 3.10 at pH 6.5 and 3.82 at pH 7.4 |
| coefficient | |
| Stability | Compound 1-(4S) is stable up to 25° C. with protection |
| | from light. |

Compound 1, Compound 1-(4S) or Form N-1, alone or in combination with other drugs, may be used to treat depression. For example, Compound 1-(4S), may be used for treating a major depressive episode in adult patients with major depressive disorder who have experienced inadequate response to separate trials of adequate dose and duration of two antidepressants from different classes, e.g., (duloxetine and escitalopram) in the current episode. Compound 1-(4S) may also be useful in treating other disorders, e.g., eating disorders and substance abuse.

Desirably, the methods of treatment and pharmaceutical compositions containing tetrahydroisoquinolines, e.g., compounds of the invention such as, for example, Compound 1-4(S), provide effective oral bioavailability, e.g., a total blood plasma concentration profile of the respective compound of the invention, as measured by AUC at 24 hours after an initial dose of the composition, that is at least greater than about 50% of the total blood plasma concentration as measured by AUC at 24 hours of an initial dose of an orally administered solution comprising the respective compound, and/or a blood plasma concentration profile after an initial dose of the composition with a Cmax of Compound 1-(4S) after an initial dose of the composition that is at least greater than about 40% of the Cmax of an orally administered solution comprising Compound 1-(4S).

Compound 1, Compound 1-(4S) or Form N-1 may be formulated with a pharmaceutical vehicle or diluent for oral, intravenous, or subcutaneous administration. The pharmaceutical composition can be formulated in a classical manner using solid or liquid vehicles, diluents, and/or additives appropriate to the desired mode of administration. Orally, the compounds can be administered in the form of tablets, including coated tablets, capsules, granules, powders, and the like. The compounds may also be administered as a suspension using carriers appropriate to this mode of administration.

The present invention contemplates the use of any pharmaceutically acceptable ingredients, such as, for example, lubricants, disintegrants, binders, fillers (also referred to as “compression aids”), surfactants, film coatings, solubilizers, and solvents. Examples of some of these ingredients are set forth below and are described in more detail in the Handbook of Pharmaceutical Excipients, Second Edition, Ed. A. Wade and P. J. Weller, 1994, The Pharmaceutical Press, London, England. The selection and amounts of such ingredients to be used in accordance with the present invention are not critical and can be determined by one skilled in the art.

Examples of lubricants suitable for use in accordance with the invention, include but are not limited to, magnesium stearate, zinc stearate, calcium stearate, stearic acid, palmitic acid, sodium stearyl fumarate, sodium benzoate, sodium lauryl sulfate, glyceryl monostearate, glyceryl palmitostearate, hydrogenated castor oil, hydrogenated vegetable oil, mineral oil, carnauba wax, and polyethylene glycol. In accordance with the invention, ingredients also referred to as “glidants” are intended to be included within the scope of lubricants. Examples include, but are not limited to, silicon dioxide, calcium silicate, calcium phosphate and talc.

Examples of disintegrants suitable for use in accordance with the invention, but are not limited to, croscarmellose sodium, crospovidone, potato starch, pregelatinized starch, corn starch, sodium starch glycolate, microcrystalline cellulose, powdered cellulose, methylcellulose, carboxymethylcellulose calcium, carboxymethylcellulose sodium, alginic acid, colloidal silicon dioxide, guar gum, magnesium aluminum silicate, polyacrilin potassium and sodium alginate.

Examples of binders suitable for use in accordance with the invention, but are not limited to, acacia, carbomer, dextrin, gelatin, guar gum, hydrogenated vegetable oil, methylcellulose, ethyl cellulose, cellulose acetate, hydroxyethyl cellulose, hydroxypropyl cellulose, hydroxypropyl methylcellulose, carboxymethylcellulose sodium, glucose, lactose, magnesium aluminum silicate, maltodextrin, polymethacrylates, povidone, polyvinyl pyrrolidone, corn starch, pregelatinized starch, alginic acid, sodium alginate, zein, carnauba wax, paraffin, spermaceti, polyethylenes and microcrystalline wax.

Examples of fillers suitable for use in accordance with the invention, but are not limited to, microcrystalline cellulose, lactose, sucrose, starch, pregelatinized starch, dextrose, dextrates, dextrin, mannitol, fructose, xylitol, sorbitol, corn starch, modified corn starch, inorganic salts such as calcium carbonate, magnesium carbonate, magnesium oxide, calcium phosphate, dicalcium phosphate, tribasic calcium phosphate, calcium sulfate, dextrin/dextrates, maltodextrin, compressible sugars, confectioner's sugar, glyceryl palmitostearate, hydrogenated vegetable oil, kaolin, maltodextrin, polymethacrylates, potassium chloride, sodium chloride, sucrose, sugar spheres and talc.

Examples of solubilizers suitable for use in accordance with the present invention include, but are not limited to, medium-chain fatty acid triglycerides, combinations of mono, di and triglycerides of long-chain fatty acids; mono- and di-long-chain fatty acid esters of polyethylene glycol (commonly known as polyoxyethylated glycerides, i.e., oleoyl polyoxylglycerides and linoleoyl polyoxylglycerides); glycerol monocaprylocaprate, glycerol monocaprylate, glycerol mono/dicaprate, and the propylene glycol mono- and di-esters of medium-chain fatty acids such as propylene glycol monocaprylate, propylene glycol monolaurate, propylene glycol dilaurate and, and combinations thereof. Examples of surfactants include polyoxyethylene sorbitan monooleate; polyoxyethylated glycerides such as polyoxyl 35 castor oil and polyoxyl 40 hydrogenated castor oil; caprylocaproyl polyoxylglycerides (medium-chain fatty acid esters of polyethylene glycol 400 and medium-chain fatty acid esters of polyethylene glycol 300); and vitamin E TPGS (i.e., d-α-tocopheryl polyethylene glycol 1000 succinate.

The formulation solutions described herein may contain one or more of various flavoring agents (e.g., cherry, berry, mint, vanilla, and the like) and/or sweetening agents (e.g., sucrose, sorbitol, mannitol, fructose, dextrose, saccharin, aspartame, acesulfame potassium, and the like) to enhance palatability of the dosage form.

The effective amount of the compounds, e.g., Compound 1, Compound 1-(4S) or Form N-1, for treating a condition may be determined by one of ordinary skill in the art, and includes exemplary dosage amounts for a mammal of from about 0.05 to about 300 mg/kg/day, preferably less than about 200 mg/kg/day, in a single dose or in 2 to 4 divided doses. In particular, doses in the range of 0.10 mg to 2.0 mg are preferred for human patients, with doses of 0.25 mg, 0.5 mg and 1.0 mg being especially preferred. It will be understood that the specific dose level and frequency of dosage for any particular subject may be varied and will depend upon a variety of factors, the bioavailability of the compounds, the metabolic stability and length of action of the compounds, the species, age, body weight, general health, sex, and diet of the subject, the mode and time of administration, rate of excretion, drug combination, and severity of the particular condition. Preferred subjects for treatment include animals, most preferably mammalian species such as humans and domestic animals such as dogs, cats, horses, and the like. Typically, the patients to whom the the compositions of the present invention may be administered will be adult or pediatric humans.

The present invention is intended to include all isotopes of atoms occurring in the compounds disclosed herein, e.g., Compound 1. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

### EXAMPLES

The following examples are provided for illustrative purposes and are not intended to limit the scope of the claimed invention.

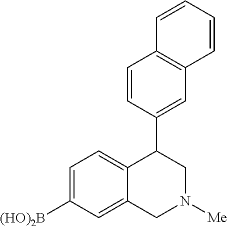 [SMILES: CN1Cc2cc(B(O)O)ccc2C(c2ccc3ccccc3c2)C1]

### Preparation of (2-methyl-4-(naphthalen-2-yl)-1,2,3,4-tetrahydroisoquinolin-7-yl)boronic acid (Compound 6)

DCM (994.2 kg) was charged into a dry glass-lined reactor at 20° C. under a nitrogen atmosphere, followed by the addition of Compound 23 (49.5 kg) and Compound 25 (38.5 kg) at 20° C. The mixture was stirred for 15 min, and then glacial acetic acid (23.0 kg) was added into the mixture at 20° C. The mixture was stirred for 45 min at 15-25° C. NaBH(OAc)₃ (89.3 kg) was then added into the reaction mixture in 5 portions at 15˜25° C. in intervals of 20˜60 min. After 2 h the reaction mixture containing 2-(3-(((2-hydroxy-2-(naphthalen-2-yl)ethyl)(methyl)amino)methyl)phenyl)-6-methyl-1,3,6,2-dioxazaborocane-4,8-dione, Compound 16 (ESI MS m/z 447) [M+H]⁺), was cooled to 0-10° C. and an aqueous solution of potassium bi-carbonate (20%, 680 kg) was added at such a rate that the reaction temperature was controlled to 0-10° C. The phases were allowed to split and the organic phase was transferred to a glass-lined reactor and treated with activated molecular sieves (4 Å) for 30 min. The suspension was then filtered and the solid residue was washed with DCM (40.0 kg). The filtrate was slowly added to a glass-lined reactor containing DCM (333 kg) and MSA (460.1 kg). The addition was done at such a rate that the internal temperature did not exceed 5° C., and stirring was maintained for an additional 10 hours. The reaction mixture containing Compound 10 (ESI MS m/z 429) [M+H]⁺) and its regiosisomer in a 10:1 ratio, was then transferred to a glass-lined reactor containing aqueous potassium bicarbonate (20%, 3000 kg) to neutralize the MSA. The transfer was done in such a way that the internal temperature was kept below 10° C. and the CO₂ off-gassing was adequately vented. After completing the addition the mixture was stirred for 45 minutes and then the phases were allowed to split. The organic phase was collected and was treated with an aqueous solution of sodium bicarbonate (10, 1361 kg) and methanol (200 kg). The biphasic mixture was stirred for 12 hours at 20° C. The phases were allowed to split and the organic phase was washed with brine (20%, 200 kg) at 20° C. The phases were allowed to split and the organic phase containing Compound 6 (ESI MS m/z 318) [M+H]⁺) was then distilled under reduced pressure to a volume of 200-250 L, followed by the addition of n-heptane (400 kg) at 20° C. to precipitate the product. The slurry was then filtered; the solid was rinsed with additional heptanes (40 kg), and was then dried at 30° C. under nitrogen to provide 50 kg (83% yield, 82% purity) of crude Compound 6 as an off-white powder.

Note: Eaton's reagent may be used as substitute of MSA

A purified sample for Compound 6 was isolated as its HCl salt IPA solvate. ¹H-NMR (500 MHz, DMSO-d₆): δ (ppm) 8.15 (s, 1H), 7.92-7.87 (m, 4H), 7.68-7.52 (m, 4H), 7.31 (d, J=7.5 Hz, 1H), 6.74 (d, J=7.6 Hz, 1H), 4.85 (m, 1H), 4.61-4.42 (m, 2H), 3.79-3.76 (m, 1H), 3.68-3.64 (m, 1H), 2.93 (s, 3H), 1.04 (d, J=6.3 Hz, 6H). ¹³C-NMR (126 MHz, DMSO-d₆): δ (ppm) 138.1, 136.7, 133.3, 133.1, 133.0, 132.2, 128.5, 128.2, 127.6, 127.6, 126.6, 126.4, 126.1, 63.9, 62.0, 55.7, 53.8, 25.4, 24.4.

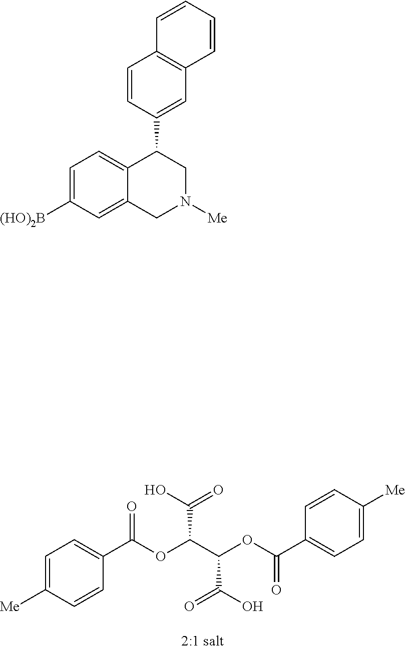 [SMILES: CN1Cc2cc(B(O)O)ccc2[C@H](c2ccc3ccccc3c2)C1 ; Cc1ccc(C(=O)O[C@H](C(=O)O)[C@H](OC(=O)c2ccc(C)cc2)C(=O)O)cc1]

### Preparation of (2S,3S)-2,3-bis((4-methylbenzoyl)oxy)succinic acid salt of (S)-(2-methyl-4-(naphthalen-2-yl)-1,2,3,4-tetrahydroisoquinolin-7-yl)boronic acid (1:2) (Compound 5)

ACN (521 kg) and water (165 kg) were charged into a glass-lined reactor under a nitrogen atmosphere at 20° C. and the mixture was stirred. Crude Compound 5 (50 kg) was then added, and the resulting mixture was heated to 70° C. A solution of D-DTTA (36.5 kg) in ACN (74 kg) and H₂O (20 kg) was then slowly added at 70° C. After ˜30% of the solution had been added, the addition was paused, seed crystals of Compound 5 (0.3 kg) were added and then the addition of the D-DTTA solution was resumed. After completion of the addition the reaction mixture was cooled to 0° C. and held for 5 hours. The slurry was filtered and the solid was sequentially washed with a mixture of ACN (108 kg) and water (34 kg) at 0° C. and with n-heptane (136 kg). The solid was then dried at 40° C. under reduced pressure providing Compound 5 as a white solid (25 kg, 31% yield, 93% purity, 96.2% e.e.). ¹H-NMR (500 MHz, DMSO-d₆): δ (ppm) 7.91-7.81 (m, 5H), 7.71 (s, 1H), 7.59 (s, 1H), 7.52-7.47 (m, 3H), 7.31 (d, J=7.9 Hz, 2H), 7.24 (d, J=7.3 Hz, 1H), 6.68 (d, J=7.9 Hz, 1H), 5.70 (s, 1H), 4.54-4.51 (m, 1H), 4.16-4.09 (m, 1H), 3.38-3.35 (m, 1H), 3.15-3.11 (m, 1H), 2.63 (s, 3H), 2.33 (s, 3H); ¹³C NMR (126 MHz, DMSO-d₆): δ (ppm) 169.3, 165.2, 143.9, 140.2, 137.5, 133.0, 132.8, 132.4, 132.1, 130.8, 129.5, 129.4, 128.2, 127.9, 127.8, 127.6, 127.0, 127.0, 126.3, 126.0, 73.1, 58.1, 55.7, 43.6, 43.3, 21.3.

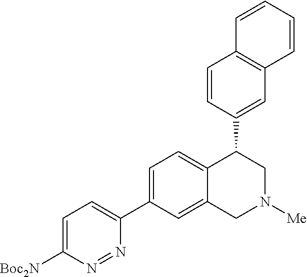

### Preparation of (S)-di-tert-butyl-(6-(2-methyl-4-(2-naphthyl)-1,2,3,4-tetrahydro-7-isoquinolinyl)-3-pyridazinyl)imidodicarbonate (Compound 28)

A solution of Pd(OAc)₂ (0.040 kg) and xanthphos (0.102 kg) in THF (3.2 kg) was added to a reactor under a nitrogen atmosphere containing a stirred solution of Compound 5 (3.60 kg), Compound 29 (2.56 kg) and potassium carbonate (3.984 kg) in THF (21.4 kg) and water (10.8 kg). The reaction mixture was heated to 55° C. for 6 hours and then cooled to 20° C. DCM (47.9 kg) and water (18 kg) were added to the reactor. The mixture was stirred for 30 min and then phases were allowed to split. The organic phase was collected and sequentially washed with water (18 kg) and brine (20%, 42.1 kg). The organic phase was pumped through a 10 micron in line filter and then azeotropically dried under reduced pressure. The volume was adjusted to ˜36 L with additional DCM and the resulting solution was pumped around through a filter containing Siliabond thiol resin (2.97 kg) for 8 hours at 20° C., and then through a 1 micron in line filter. The reaction mixture was then solvent exchange into toluene and adjusted to a volume of approximately 43 L under reduced pressure and the resulting slurry was heated to 60° C. MeOH was added (3.8 kg) and the resulting solution was cooled to 40° C. Approximately 6.5 L were distilled under a reduced pressure and the mixture was cooled to 20° C. over 3 hours and held for an additional 1 hour. The slurry was then filtered and the solid sequentially washed with MeOH (13.9 kg) and n-heptane (12.0 kg). The solid was dried under reduced pressure with a nitrogen sweep at 50° C. affording Compound 28 as a white solid in 67% yield (2.2 kg, 98.7% purity, 99% e.e.). ¹H NMR (CDCl3, 400 MHz) δ (ppm) 7.98 (d, J=1.6 Hz, 1H); 7.88 (d, J=8.9 Hz, 1H); 7.857.76 (m, 3H); 7.74-7.70 (m, 2H); 7.52 (d, J=8.9 Hz, 1H); 7.51-7.44 (m, 2H); 7.30 (dd, J=8.5, 1.6 Hz, 1H); 7.06 (d, J=7.9 Hz, 1H); 4.52 (m, 1H); 3.93 (d, J=14.9 Hz, 1H); 3.76 (d, J=14.9 Hz, 1H); 3.16 (dd, J=11.7, 5.7 Hz, 1H); 2.72 (dd, J=11.7, 8.8 Hz, 1H); 2.50 (s, 3H); 1.47 (s, 18H). ¹³C NMR (CDCl₃, 100 MHz) δ (ppm) 157.7, 154.8, 150.4, 141.5, 139.5, 136.2, 133.3, 133.3, 132.3, 130.1, 128.0, 127.7, 127.5, 127.5, 127.0, 126.0, 125.8, 125.5, 125.0, 124.8, 83.8, 61.3, 58.3, 45.9, 45.8, 27.7. ESI MS m/z 567 [M+H]⁺. [α]_{D}²²=+102.1 (c=0.01 g/mL, DCM).

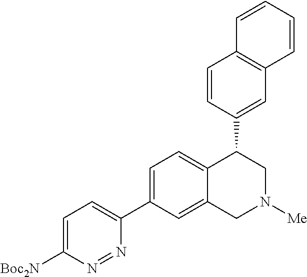

### Preparation of (S)-di-tert-butyl-(6-(2-methyl-4-(2-naphthyl)-1,2,3,4-tetrahydro-7-isoquinolinyl)-3-pyridazinyl)imidodicarbonate (Compound 28)

Under a nitrogen atmosphere, THF (97.4 L), water (91.5 L) and potassium carbonate (19.8 kg) were added to a glass lined reactor at 20° C. and the resulting solution was stirred and inerted to remove oxygen. Compound 5 (12.2 kg) and Compound 29 (8.7 kg) were charged and the resulting solution was stirred and inerted to remove oxygen. PdCl₂ (xanthphos) (0.181 kg) was then added followed by a THF rinse (24.3 L), and the resulting solution was heated to 55-60° C. and agitated for 3 hours. After reaction completion, the reaction mixture was cooled to 40° C. within 30 min and then reheated to 53° C. to establish a seed bed. IPA (91.5 L) was charged at 53° C. over 1 hour and the resulting slurry was cooled to 20° C. over 2 hours. The solid product was filtered and washed with 50% (v/v) aqueous IPA (97.6 L) and then with IPA (48.8 L). After drying at reduced pressure at 50-60° C., Compound 28 was isolated as a white solid (10.9 kg, 80% yield, >99% purity, >99.95% e.e.). ¹H NMR (CDCl3, 400 MHz) δ (ppm) 7.98 (d, J=1.6 Hz, 1H); 7.88 (d, J=8.9 Hz, 1H); 7.857.76 (m, 3H); 7.74-7.70 (m, 2H); 7.52 (d, J=8.9 Hz, 1H); 7.51-7.44 (m, 2H); 7.30 (dd, J=8.5, 1.6 Hz, 1H); 7.06 (d, J=7.9 Hz, 1H); 4.52 (m, 1H); 3.93 (d, J=14.9 Hz, 1H); 3.76 (d, J=14.9 Hz, 1H); 3.16 (dd, J=11.7, 5.7 Hz, 1H); 2.72 (dd, J=11.7, 8.8 Hz, 1H); 2.50 (s, 3H); 1.47 (s, 18H). ¹³C NMR (CDCl₃, 126 MHz) δ (ppm) 157.7, 154.8, 150.4, 141.5, 139.5, 136.2, 133.3, 133.3, 132.3, 130.1, 128.0, 127.7, 127.5, 127.5, 127.0, 126.0, 125.8, 125.5, 125.0, 124.8, 83.8, 61.3, 58.3, 45.9, 45.8, 27.7. ESI MS m/z 567 [M+H]⁺. [α]_{D}²²=+102.1 (c=0.01 g/mL, DCM).

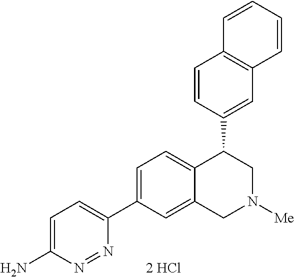 [SMILES: CN1Cc2cc(-c3ccc(N)nn3)ccc2[C@H](c2ccc3ccccc3c2)C1]

### (S)-6-(2-methyl-4-(naphthalen-2-yl)-1,2,3,4-tetrahydroisoquinolin-7-yl)pyridazin-3-amine-bis-hydrochloride salt (Compound 24)

HCl in IPA (5-6 N, 8.53 kg) was added to a stirred suspension of Compound 28 (1.25 kg) in wet IPA (5.1 kg, 5% water content) at 20° C. in a glass-lined reactor under a nitrogen atmosphere. The resulting mixture was heated to 70° C. and aged for 2 h and then cooled to 20° C. IPAc (21.75 kg) was added and the slurry was stirred for no less than 1 h. The slurry was filtered and then sequentially washed first with a mixture of IPA (1.68 kg) and IPAc (3.60 kg) and secondly with pure IPAc (5.44 kg). The solid was then dried under reduced pressure at 50° C. yielding Compound 24 as a white solid (0.88 kg, 93%, >99% purity, >99.95% e.e.). ¹H NMR (500 MHz, DMSO-d6) δ (ppm)=12.19 (br. s, 1H), 8.99 (br. s, 1H), 8.37 (d, J=9.8 Hz, 1H), 8.00-7.85 (m, 5H), 7.79-7.66 (m, 2H), 7.58-7.48 (m, 2H), 7.32 (br. s., 1H), 6.98-6.82 (m, 1H), 5.01-4.87 (m, 1H), 4.82-4.56 (m, 2H), 3.90-3.82 (m, 1H), 3.82-3.75 (m, 1H), 2.98 (s, 3H).

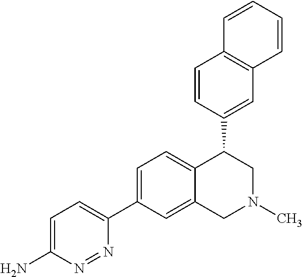 [SMILES: CN1Cc2cc(-c3ccc(N)nn3)ccc2[C@H](c2ccc3ccccc3c2)C1]

### Preparation of (S)-6-(2-methyl-4-(naphthalen-2-yl)-1,2,3,4-tetrahydroisoquinolin-7-yl)pyridazin-3-amine (Compound 1-(4S))

Compound 24 (0.88 kg) was dissolved MeOH (25.36 kg) and water (8.76 kg) and the resulting solution was pumped through a 1 micron in-line filter. The mixture was stirred, heated to 50° C. and an aqueous solution of NaOH was slowly added. After addition of 2.07 kg of aqueous NaOH the reaction was seeded with Compound 1-(4S) (0.008 kg) and held for no less than 10 min. Additional aqueous NaOH was added (4.15 kg) and the reaction mixture was cooled to 20° C. over 30 min and then held for 1 h. The slurry was filtered and sequentially washed three times with 50% aqueous MeOH (v/v) (3.94 kg) and finally with IPA (3.46 kg). The solid was dried under reduced pressure at 50° C. yielding Compound 1-(4S) as a white solid (0.71 kg, 95%, >99% purity, >99.95% e.e.). ¹H NMR (500 MHz, DMSO-d6) δ (ppm) 7.88-7.80 (m, 3H), 7.79-7.74 (m, 3H), 7.65 (d, J=8.2 Hz, 1H), 7.50-7.43 (m, 2H), 7.36 (dd, J=8.5, 1.6 Hz, 1H), 6.89 (d, J=8.2 Hz, 1H), 6.85 (dd, J=9.3, 1.4 Hz, 1H), 6.49 (s, 2H), 4.41 (t, J=6.3 Hz, 1H), 3.76-3.66 (m, 2H), 2.97 (dd, J=11.3, 5.7 Hz, 1H), 2.67 (dd, J=11.3, 7.3 Hz, 1H), 2.34 (s, 3H). ¹³C NMR (126 MHz, DMSO-d6) δ (ppm) 159.7, 149.6, 142.6, 137.0, 135.7, 134.8, 132.9, 131.8, 129.5, 127.7, 127.5, 127.4, 127.3, 127.2, 126.0, 125.5, 125.2, 123.3, 123.1, 114.1, 60.7, 57.8, 45.6, 44.7. ESI MS m/z 367 [M+H]⁺.

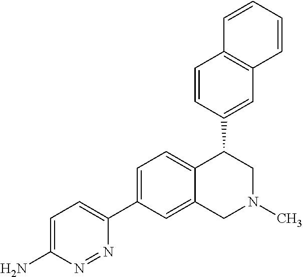 [SMILES: CN1Cc2cc(-c3ccc(N)nn3)ccc2[C@H](c2ccc3ccccc3c2)C1]

### Preparation of (S)-6-(2-methyl-4-(naphthalen-2-yl)-1,2,3,4-tetrahydroisoquinolin-7-yl)pyridazin-3-amine (Compound 1-(4S))

A stirred suspension of Compound 26 (10 g) in MeOH (30 mL) and water (30 mL) was heated to 63° C. Concentrated aqueous HCl was added and the resulting mixture was heated to 70° C. and stirred for 2 h. The reaction mixture was cooled to 20° C., additional MeOH was added (30 mL) and was then the solution was filtered through a 0.45 micron Teflon ZapCap. The solution was heated to 40° C. and aqueous 1N NaOH was slowly added. After addition of 14.92 mL the reaction was seeded with crystals of Compound 1-(4S) (0.065 g) and aged for 15 min. Additional aqueous 1N NaOH (28.76 mL) was added and the reaction mixture was then cooled to 20° C. The slurry was filtered and the solid was sequentially washed with pre-mixed MeOH/H₂O (50/50 v/v, 80 mL) and IPA (30 mL). The solid was dried under reduced pressure at 50° C. yielding Compound 1-(4S) as a white solid (6.2 g, 96%, >99% purity, >99.95% e.e.). ¹H NMR (500 MHz, DMSO-d6) δ (ppm) 7.88-7.80 (m, 3H), 7.79-7.74 (m, 3H), 7.65 (d, J=8.2 Hz, 1H), 7.50-7.43 (m, 2H), 7.36 (dd, J=8.5, 1.6 Hz, 1H), 6.89 (d, J=8.2 Hz, 1H), 6.85 (dd, J=9.3, 1.4 Hz, 1H), 6.49 (s, 2H), 4.41 (t, J=6.3 Hz, 1H), 3.76-3.66 (m, 2H), 2.97 (dd, J=11.3, 5.7 Hz, 1H), 2.67 (dd, J=11.3, 7.3 Hz, 1H), 2.34 (s, 3H). ¹³C NMR (126 MHz, DMSO-d6) δ (ppm) 159.7, 149.6, 142.6, 137.0, 135.7, 134.8, 132.9, 131.8, 129.5, 127.7, 127.5, 127.4, 127.3, 127.2, 126.0, 125.5, 125.2, 123.3, 123.1, 114.1, 60.7, 57.8, 45.6, 44.7. ESI MS m/z 367 [M+H]⁺.

### Example A

### API (Active Pharmaceutical Ingredient)

The following discussion provides an example of the determination of ppm amounts of impurities in a sample of the API (Compound 1). The example focuses on Compound 3. Other impurities are determined in an analogous way.

The determination of the ppm levels of Compound 3 in Compound 1 is done using common practices for those skilled in the art of Analytical Chemistry. A working standard of Compounds 3 of known purity is required. The analysis is conducted in a HPLC instrument using a validated analytical method (For validation of analytical methods in pharmaceutical applications, see: “Validation of Analytical Procedures: Text and Methodology; ICH Guidelines Q2(R1)” and according to standard analysis practices.

A Working Standard Solution is prepared by dissolving Compound 3 in mixtures of MeOH, ACN, water and TFA in a volumetric flask using a reference standard of Compound 3 of known purity. Ultrasounds are used to aid the dissolution process.

A Working Sample Solution is prepared by dissolving Compound 1 in mixtures of MeOH, ACN, water and TFA in a volumetric flask. Ultrasounds are used to aid the dissolution process.

The blank, the sample suitability, the working standard solution and the working sample solution are analyzed by HPLC.

HPLC Parameters

Column: Waters Symmetry C18, 150 mm×4.6 mm i.d., 3.5 μm particle size.

Equivalent may be substituted.

Detector Wavelength: 225 nm Column Temperature: 25° C.

Injection Volume: 8 μL Flow Rate: 1.0 mL/minute

Run Time: ˜40 minutes Sample Temperature: 5° C.

Gradient Program Listing:

| | | |
|---|---|---|
| Time | Mobile Phase Composition | Gradient |

| (minutes) | % A | % B | Profile |
|---|---|---|---|
| 0 | 85 | 15 | Initial |
| 10 | 60 | 40 | Linear |
| 15 | 50 | 50 | Linear |
| 30 | 50 | 50 | Isocratic |
| 31 | 85 | 15 | Initial |

Calculations:

The amount (ppm) of Compound 3 in Compound 1 is calculated using the following formula:

C_{G}/C_{A}

In which C_{G} is the concentration (ng/mL) of Compound 3 determined in the Working Sample Solution and C_{A} is the concentration (mg/mL) of Compound 1 in the Working Sample Solution.

The concentration of Compound 1 in the Working Sample Solution, C_{A}, is calculated using weight and volume in following formula:

Wu/Vu

Wu is the weight (mg) of Compound 1 in the Working Sample Solution and Vu is the total volume (mL) of the Working Sample Solution. The concentration of Compound 1 in the Working Sample Solution is expressed in terms of its salt free form.

The concentration of Compound 3 in the Working Sample Solution, C_{G}, is calculated using the following formula:

Cs(ru/rs)

In which Cs is the concentration (ng/mL) of compound 3 in the Working Standard Solution and

ru and rs are the peak responses (expressed as area counts) obtained by integration of the HPLC chromatograms of the Working Sample Solution and Working Standard Solution, respectively.

The concentration of Compound 3 in the Working Standard Solution, Cs, is calculated using the weight and volume, while applying the purity value of the reference standard. The purity of the reference standard is calculated such that the purity value is expressed in terms of Compound 3 in its salt free form and accounting for any water or solvent content. The concentration of the Working Standard

Solution, Cs, is calculated as follows:

[Ws×P]/Vs

Ws is the weight (mg) of the reference standard for Compound 3 in the Working Standard Solution, P is the purity of reference standard (expressed as a fraction), Vs is the volume (mL) of the Working Standard Solution.

Drug Product

The following discussion provides an example of the determination of ppm amounts of impurities in a sample of the Drug Product (Formulated API). The example focused on Compound 3. Other impurities will be determined in an analogous way.

The determination of the ppm levels of Compound 3 in the Drug Product is done using common practices for those skilled in the art of Analytical Chemistry. A working standard of Compounds 3 of known purity is required. The analysis is conducted in a HPLC instrument using a validated analytical method (For validation of analytical methods in pharmaceutical applications, see: “Validation of Analytical Procedures: Text and Methodology; ICH Guidelines Q2(R1)” and according to standard analysis practices.

A Working Standard Solution is prepared by dissolving Compound 3 in mixtures of MeOH, ACN, water and TFA in a volumetric flask using a reference standard of Compound 3 of known purity. Ultrasounds are used to aid the dissolution process.

A Working Sample Solution is prepared by dissolving several tablets of drug product in mixtures of MeOH, ACN, water and TFA in a volumetric flask. Ultrasounds are used to aid the dissolution process.

The blank, the sample suitability, the working standard solution and the working sample solution are analyzed by HPLC.

HPLC Parameters

Column: Waters Symmetry C18, 150 mm×4.6 mm i.d., 3.5 μm particle size.

Equivalent may be substituted.

Detector Wavelength: 225 nm Column Temperature: 25° C.

Injection Volume: 8 μL Flow Rate: 1.0 mL/minute

Run Time: ˜40 minutes Sample Temperature: 5° C.

Gradient Program Listing:

| | | |
|---|---|---|
| Time | Mobile Phase Composition | Gradient |

| (minutes) | % A | % B | Profile |
|---|---|---|---|
| 0 | 85 | 15 | Initial |
| 10 | 60 | 40 | Linear |
| 15 | 50 | 50 | Linear |
| 30 | 50 | 50 | Isocratic |
| 31 | 85 | 15 | Initial |

Calculations:

The amount (ppm) of Compound 3 in the Drug Product is calculated using the following formula:

C_{G}/C_{A}

In which C_{G} is the concentration (ng/mL) of Compound 3 determined in the Working Sample Solution and C_{A} is the concentration (mg/mL) of Compound 1 in the Working Sample Solution. The concentration of Compound 1 in the Working Sample Solution, C_{A}, is calculated using weight and volume in following formula:

(T_{N}*Tₛ)/Vu

Where T_{N} is the number of drug product tables used in the preparation of the Working Sample Solution, Tₛ is their tablet strength (mg of Compound 1 per tablet of Drug product) and Vu is the total volume (mL) of the Working Sample Solution. The concentration of Compound 1 in the Working Sample Solution is expressed in terms of its salt free form.

The concentration of Compound 3 in the Working Sample Solution, C_{G}, is calculated using the following formula:

Cs(ru/rs)

In which Cs is the concentration (ng/mL) of Compound 3 in the Working Standard Solution and ru and rs are the peak responses (expressed as area counts) obtained by integration of the HPLC chromatograms of the Working Sample Solution and Working Standard Solution, respectively.

The concentration of Compound 3 in the Working Standard Solution, Cs, is calculated using the

weight and volume, while applying the purity value of the reference standard. The purity of the reference standard is calculated such that the purity value is expressed in terms of Compound 3 in its salt free form and accounting for any water or solvent content. The concentration of the Working Standard

Solution, Cs, is calculated as follows:

[Ws×P]/Vs

Ws is the weight (mg) of the reference standard for Compound 3 in the Working Standard Solution, P is the purity of reference standard (expressed as a fraction), Vs is the volume (mL) of the Working Standard Solution.

### Example B

### Preparation of Tablets

A 0.5% common granulation blend was prepared by spraying a solution of Compound 1-(4S) in 0.1 M citric acid solution with sulfobutyl β-cyclodextrin to the excipient blend consisting of microcrystalline cellulose, mannitol, pregelatinized starch followed by drying and milling using a one pot processor. Then, the blend was mixed with magnesium stearate to form a common granulation for tablet compression. Various tablet strengths were manufactured by varying tablet press weight. For example, 0.25 mg, 0.5 mg and 1 mg strength tablets were manufactured by compressing the common granulation at 50 mg, 100 mg and 200 mg press weight, respectively. The tablets were coated with Opadry II white. Opadry II white contains polivinyl alcohol, titanium dioxide, polyethylene glycol, 3350, and talc. The resulting tablets were white to off-white round film-coated tablets containing 0.25-mg, 0.5-mg and 1-mg of Compound 1-(4S) and the following inactive ingredients: microcrystalline cellulose, mannitol, pregelatinized starch, citric acid monohydrate, beta-cyclodextrin sulfobutyl ether sodium salt, magnesium stearate, and Opadry® II white.

### Example C

### In Vitro Studies

Competition binding experiments were performed to determine the in vitro binding potencies (IC50 values) of Compound 1-(4S) for SERT, DAT, and NET. Membranes prepared from human embryonic kidney (HEK) cells overexpressing each transporter were employed. Radioligands were used at their respective affinity constant (Kd) concentrations for each transporter to enable direct comparisons of the IC50 values between assays. Compound 1-(4S) completely saturated SERT, DAT, and NET binding sites with IC50 values of 1.08±0.09 nM (n=42), 5.67±0.36 nM (n=42), and 7.99±0.43 nM (n=37), respectively. The N-demethylated metabolite of Compound 1-(4S), Compound 31 (shown below), completely inhibited binding to SERT, DAT, and NET with IC50 values of 0.21±0.04 nM (n=11), 6.19±1.0 nM (n=11), and 26.7±3.0 nM (n=11), respectively. The activity of the metabolite at off-target sites was similar to the parent, with activity at the 5-HT1A (inhibition constant [Ki]=99 nM), adrenergic α1A (Ki=410 nM), α1B (Ki=260 nM) and α1D (Ki=480 nM) receptors.

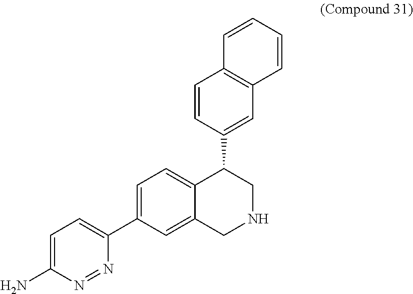 [SMILES: Nc1ccc(-c2ccc3c(c2)CNC[C@H]3c2ccc3ccccc3c2)nn1]

Cell-based assays were used to determine the ability of Compound 1-(4S) to inhibit reuptake of 3H-labeled DA ([3H]DA), NE ([3H]NE), and 5-HT ([3H]5-HT). Compound 1-(4S) potently and completely inhibited uptake of [3H]DA into HEK293/hDAT cells with an IC50 of 31.5±2.5 nM (n=2), [3H]NE into HEK293/hNET cells with an IC50 of 37.5±2.5 nM (n=2), and [3H]5-HT into HEK293/hSERT cells displaying an IC50 of 18.5±5.5 nM (n=2).

Results from in vitro nonclinical pharmacology studies were as follows:

### In Vitro Binding: Compound 1-(4S)

SERT binding IC50=1.08±0.09 nM
Number of Repeats of Assay 42
DAT binding IC50=5.67±0.36 nM
Number of Repeats of Assay 42
NET binding IC50=7.99±0.43 nM
Number of Repeats of Assay 37

### In Vitro Binding: Compound 31

SERT binding IC50=0.21±0.04 nM
Number of Repeats of Assay 11
DAT binding IC50=6.19±1.0 nM
Number of Repeats of Assay 11
NET binding IC50=26.7±3.0 nM
Number of Repeats of Assay 11

### Monoamine Reuptake Inhibition Compound 1-(4S)

Serotonin reuptake IC50=18.5±5.5 nM
Number of Repeats of Assay 2
Dopamine reuptake IC50=31.5±2.5 nM
Number of Repeats of Assay 2
Norepinephrine reuptake IC50=37.5±2.5 nM
Number of Repeats of Assay 2
   Preferably, the compositions of the present invention can lead to greater than about 10% DAT occupancy, e.g., 10-40%, while maintaining SERT occupancy greater than about 60%, e.g., 60-80%.

### Example D

### Pharmacodynamics

The effects of Compound 1-(4S) in the mouse tail suspension model of depression and target site occupancy were assessed to compare occupancies associated with the behavioral response. The mouse tail suspension model is known the art. Compound 1-(4S) dose-dependently decreased immobility in the model. The lowest effective dose of Compound 1-(4S), 0.3 mg/kg, PO, decreased immobility by 77% relative to vehicle-treated controls. A higher dose of Compound 1-(4S) (1.0 mg/kg, PO) also significantly decreased immobility by 91%. Administration of Compound 1-(4S) led to dose-dependent occupancy of SERT, NET, and DAT binding sites in the brain. At the lowest behaviorally-effective dose (0.3 mg/kg), occupancies were 86±1% of SERT, 76±3% of NET, and 28±3% of DAT. At 1 mg/kg, occupancies were 89±1%, 82±1% and 54±3% at SERT, NET, and DAT, respectively. Pharmacokinetics and pharmacodynamics (PD) in mouse, rat, and dog focused primarily on DAT occupancy since SERT and NET occupancies were saturated by drug for most, if not all, of the times studied. DAT occupancy increased as a function of increasing plasma exposure. The DAT occupancy to total plasma level could be fitted using a single binding site model and plasma level ED50 (the dose producing 50% effect) values could be calculated. For the mouse, rat, and dog, the total plasma concentrations producing 50% effect (EC50s) values were 1193 nM, 47 nM, and 103 nM, respectively.

To better understand the margin between doses of Compound 1-(4S) that are effective in the mouse tail suspension models and those that lead to monoamine-related side effects, locomotor activity was examined to assess psychostimulant properties and stereotypy was examined to assess more severe DA-related side effects. Increased body temperature, another effect that has been attributed to DA pharmacology, was also studied. In the mouse, Compound 1-(4S) increased locomotor activity and stereotypy scores at doses of 3.0 mg/kg and 10 mg/kg, respectively. Thus, the margin between the Compound 1-(4S) effective dose in the mouse tail suspension and increased locomotor activity was 10-fold and the margin versus activity in stereotypy was 30-fold. The thermogenic effect of Compound 1-(4S) was studied in mice and rats by measuring changes in rectal temperature following administration of the compound. In the mouse, significant increases in the rectal temperature were observed 4 hours after doses of 1, 3, and 10 mg/kg of Compound 1-(4S). The onset of the thermogenesis was earlier as the dose was increased. The levels of SERT and NET occupancy were over 80% and appeared saturated across the doses tested. The DAT occupancy was dose-dependent, with 55±5.7% at 1.0 mg/kg, 74±6% at 3.0 mg/kg, and 83±0.8% at 10 mg/kg. These data, along with those generated from rats, suggest that DAT occupancy is responsible for the thermogenic effect of Compound 1-(4S). Significant increases in rectal temperature in the rat were observed with doses of 3.0 and 10 mg/kg, but not 1.0 mg/kg. The onset of the temperature increase was earlier at the highest dose of 10 mg/kg. SERT and NET occupancy was nearly saturated at all 3 doses. The DAT occupancy was 37±8.3%, 55±5.2%, and 71±1.7% at 1.0, 3.0, and 10 mg/kg, respectively. Consistent with data obtained with mice, the thermogenic effect of Compound 1-(4S) is dose dependently related to DAT occupancy and at least 50% DAT occupancy is needed for this effect.

### Example E

### Drug Discrimination Assay

Compound 1-(4S) was evaluated in a cocaine discrimination assay in rats, a model used to address abuse potential. The cocaine discrimination assay is known the art Compound 1-(4S) was administered and the percentage of rats that responded by selecting the cocaine-associated lever was measured. Compound 1-(4S) administered 60 minutes prior to the test, resulted in an inverted U-shaped curve. The dose of Compound 1-(4S) producing the peak effect (0.3 mg/kg, PO) resulted in 53% cocaine-lever responding. A lower dose of Compound 1-(4S) (0.1 mg/kg, PO) resulted in less than 1% cocaine-lever responding, and a higher dose (1.0 mg/kg, PO) resulted in 25% and 32% cocaine-lever responding on two test occasions. Since the subjective effects of cocaine are evident 15 minutes after dosing, Compound 1-(4S) was tested at the earlier time point. The dose of Compound 1-(4S)BMS-820836 that resulted in 53% cocaine-lever responding when tested 60 minutes after dosing (0.3 mg/kg, PO), resulted in only 11% cocaine-lever responding when tested at the earlier time point. Two higher doses of Compound 1-(4S) tested (1.0 and 3.0 mg/kg, PO) resulted in 10% and 28% cocaine-lever responding, respectively, when tested after 15 minutes. In summary, Compound 1-(4S) partially substituted for the cocaine stimulus in the rat discrimination model. In addition, the onset of this partial effect was slower than the onset of the subjective effects of cocaine in this model. As most drugs of abuse have a rapid onset of central nervous system (CNS) effects, the data obtained with Compound 1-(4S) suggest that this compound may have a lower abuse potential than a potent psychomotor stimulant.

While this invention has been described with an emphasis upon specific aspects, those skilled in the art will recognize that variations in the aspects disclosed may be used and that it is intended that the invention may be practiced otherwise than as specifically described herein. Accordingly, this invention includes all modifications encompassed within the spirit and scope of the invention as defined by the claims that follow. For example, it should be understood that the reaction steps set forth in the appended claims need not necessarily be performed in the order in which they appear, and one skilled in the field may be able to vary the order of reaction steps. Additionally, certain reaction sequences may be performed simultaneously, or these reactions can be performed in separate steps, without departing from the spirit and scope of the invention. It is intended that all such modifications are encompassed within the scope of the appended claims. In addition, alternative process steps or process conditions effective to promote the formation of the desired compounds, e.g., different reagents, acids, salts, protecting groups, techniques, catalysts, pressures, temperatures, reaction times, and the like, are known to those skilled in the art and may be employed and are intended to be within the scope of the claims which follow.

Where noted above, publications and references, including but not limited to patents and patent applications, cited in this specification are herein incorporated by reference in their entirety in the entire portion cited as if each individual publication or reference were specifically and individually indicated to be incorporated by reference herein as being fully set forth.

1. A composition comprising a compound having the following formula: 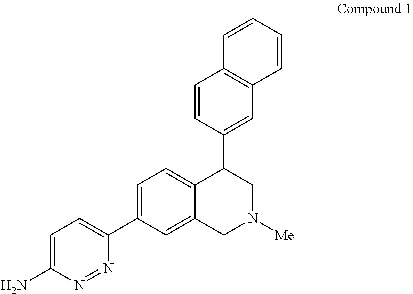 [SMILES: CN1Cc2cc(-c3ccc(N)nn3)ccc2C(c2ccc3ccccc3c2)C1]
and a compound having the following formula: 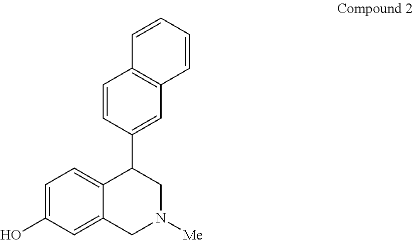 [SMILES: CN1Cc2cc(O)ccc2C(c2ccc3ccccc3c2)C1]
said Compound 2 present in an amount of from about 1 ppm to less than 1.0 wt % based on the weight of Compound 1.

2. The composition of claim 1 wherein the amount of 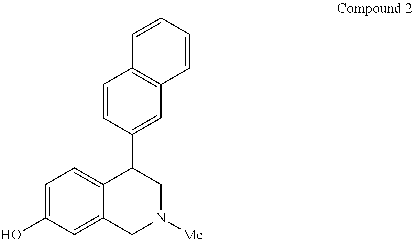 [SMILES: CN1Cc2cc(O)ccc2C(c2ccc3ccccc3c2)C1]
is less than about 1500 ppm based on the weight of Compound 1.

3. The composition of claim 2 wherein the amount of 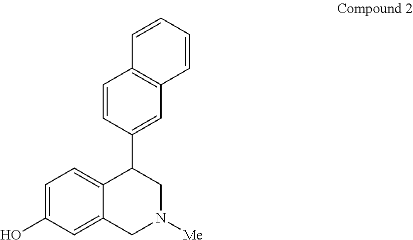 [SMILES: CN1Cc2cc(O)ccc2C(c2ccc3ccccc3c2)C1]
is less than about 500 ppm based on the weight of Compound 1.

4. A composition comprising a compound having the following formula: 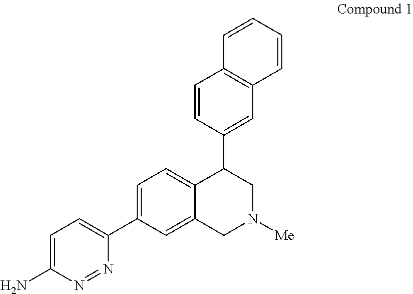 [SMILES: CN1Cc2cc(-c3ccc(N)nn3)ccc2C(c2ccc3ccccc3c2)C1]
and a compound having the following formula: 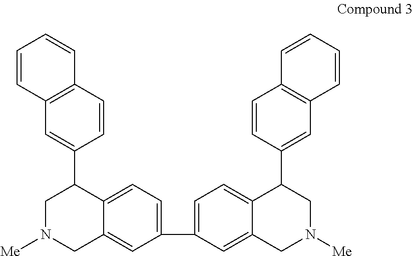 [SMILES: CN1Cc2cc(-c3ccc4c(c3)CN(C)CC4c3ccc4ccccc4c3)ccc2C(c2ccc3ccccc3c2)C1]
said Compound 3 present in an amount of from about 1 ppm to less than 1.0 wt % based on the weight of Compound 1.

5. A composition of claim 4 wherein the amount of 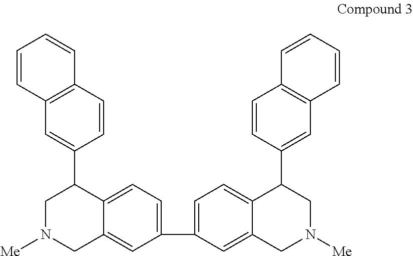 [SMILES: CN1Cc2cc(-c3ccc4c(c3)CN(C)CC4c3ccc4ccccc4c3)ccc2C(c2ccc3ccccc3c2)C1]
is less than about 1500 ppm based on the weight of Compound 1.

6. A composition of claim 5 wherein the amount of 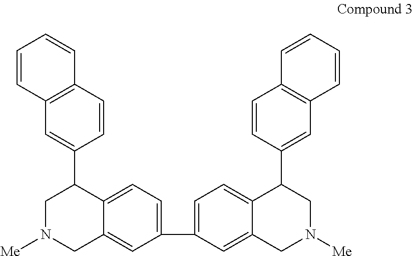 [SMILES: CN1Cc2cc(-c3ccc4c(c3)CN(C)CC4c3ccc4ccccc4c3)ccc2C(c2ccc3ccccc3c2)C1]
is less than about 500 ppm based on the weight of Compound 1.

7. A composition of claim 1 further comprising a compound having the following formula: 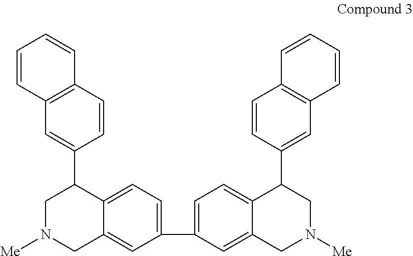 [SMILES: CN1Cc2cc(-c3ccc4c(c3)CN(C)CC4c3ccc4ccccc4c3)ccc2C(c2ccc3ccccc3c2)C1]
said Compound 3 present in an amount of from about 1 ppm to less than 1.0 wt % based on the weight of Compound 1.

8. A compound having the formula: 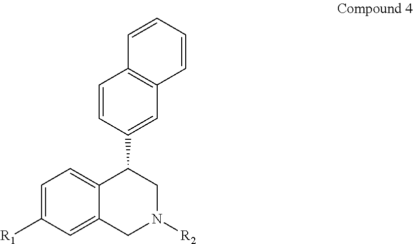
wherein
R₁ is selected from the group consisting of B(OH)₂, B(OR₃)₂, B(O₂COR₃)₂, and —BF₃K;
R₂ is selected from the group consisting of H, CH₃, CH₂CH₂SiMe₃, benzyl, diphenylmethyl, p-methoxybenzyl, 3,4-dimethoxybenzyl, COOC(CH₃)₃; COOCH₃, COOCH₂CH₃, COOAllyl, COObenzyl, COO-p-methoxybenzyl, COOCH₂CH₂SiMe₃, 9-Fluorenylmethyloxycarbonyl, COOCH₂CH₂CCl₃, COH, COMe, COPh, and COCF₃, and
R₃ is selected from the group consisting of alkyl, aryl and heteroaryl;
and/or salts thereof.

9. A composition of claim 1 wherein Compound 1 is in the form of Compound 1-(4S).

10. A composition of claim 1 wherein Compound 1 is in the crystalline form of Form N-1.

11. A composition of claim 9 in the form of a tablet or capsule comprising from about 0.10 to 2.0 mg of Compound 1-(4S).

12. A composition of claim 9 in the form of a tablet or capsule comprising from about 0.10 to 2.0 mg of Compound 1-(4S) further comprising a pharmaceutically acceptable additive.

13. A composition of claim 9 in the form of a tablet or capsule comprising from about 0.10 to 2.0 mg of Compound 1-(4S) further comprising a pharmaceutically acceptable additive, the composition providing a total blood plasma concentration profile of Compound 1-(4S), as measured by AUC at 24 hours after an initial dose of the composition, that is at least greater than about 50% of the total blood plasma concentration as measured by AUC at 24 hours of an initial dose of an orally administered solution comprising Compound 1-(4S).

14. A composition of claim 9 in the form of a tablet or capsule comprising from about 0.10 to 2.0 mg of Compound 1-(4S) further comprising a pharmaceutically acceptable additive, the composition providing a blood plasma concentration profile after an initial dose of the composition has a Cmax of Compound 1-(4S) that is at least greater than about 40% of the Cmax of an orally administered solution comprising Compound 1-(4S).

15. A method of treating depression comprising administering to a patient in need thereof the composition of claim 1.

16. A method of claim 15 wherein Compound 1 is in the form of Compound 1-(4S).

17. A method of treating a major depressive episode in adult patients with major depressive disorder who have experienced inadequate response to separate trials of adequate dose and duration of two antidepressants from different classes in the current episode, comprising administering to the patient a composition comprising Compound 1-(4S).

18. A method of treating depression comprising administering to a patient in need thereof a composition of claim 9 wherein a total blood plasma concentration profile of Compound 1-(4S) is provided, as measured by AUC at 24 hours after an initial dose of the composition, that is at least greater than about 50% of the total blood plasma concentration as measured by AUC at 24 hours of an initial dose of an orally administered solution comprising Compound 1-(4S).

19. A method of treating depression comprising administering to a patient in need thereof a composition of claim 9 wherein a blood plasma concentration profile after an initial dose of the composition has a Cmax of Compound 1-(4S) that is at least greater than about 40% of the Cmax of an orally administered solution comprising Compound 1-(4S).
